(19) United States
(12) Patent Application Publication
(10) Pub. No.: US 2016/0319325 A1
(43) Pub. Date: Nov. 3, 2016

(54) Swatch for Testing Lipase Activity

(71) Applicant: NOVOZYMES A/S, Bagsvaerd (DK)

(72) Inventors: Yuan Xu, Beijing (CN); Steffen Kayser, Copenhagen (DK); Bitten Plesner, Copenhagen (DK)

(73) Assignee: Novozymes A/S, Bagsvaerd (DK)

(21) Appl. No.: 15/107,654

(22) PCT Filed: Jan. 23, 2015
(86) PCT No.: PCT/CN2015/071402

§ 371 Date: Jun. 23, 2016

(30) Foreign Application Priority Data
Jan. 24, 2014 (CN) PCT/CN2014/071355

(51) Int. Cl. C12Q 1/44; C11D 3/386

(52) U.S. Cl. CPC C12Q 1/44; C12Y 301/01003; C11D 3/38627; G01N 2333/92

(57) ABSTRACT

The present invention provides a swatch comprising a fabric and a colored lipid stain, wherein said colored lipid stain comprises one or more of several lipase substrates, at least one carotenoid, and optionally a color preserving agent. The present invention also provides a method for preparing the swatch.

### REFERENCE TO A SEQUENCE LISTING

This application contains a Sequence Listing in computer readable form, which is incorporated herein by reference.

### TECHNICAL FIELD

The present invention relates to a swatch for testing wash performance of lipase, e.g. for use in detergent compositions.

### BACKGROUND OF THE INVENTION

Lipases are lipolytic enzymes well known as ingredient in compositions used for cleaning. During development and selection of lipases for such compositions suitable assays and test systems are required for determining and comparing wash performance. Wash performance of lipases are usually tested by subjecting fabrics containing suitable lipolytic stains in a standardized way with compositions containing the lipase to be tested.

WO02/42740 discloses an automated assay for determining the wash performance of cleaning and detergent ingredients e.g. enzymes and/or combinations of enzymes.

U.S. Pat. No. 5,023,095 discloses a color stabilization system for beta-carotene for dry food mixes and a food coloring composition comprising 0.5-5.0 wt % beta-carotene, 0.5-5.0 wt % oil and 0.05-1.5 wt % dl-alpha-tocopherol.

WO93/04598 discloses a carotenoid composition for coloring of food comprising (a) a carotenoid in an oil solvent wherein the weight of carotenoid in the carotenoid composition is up to 12%; (b) a dispersion of a water dispersible matrix and a stabiliser, and optionally a non-oil solvent; and (c) an emulsifier; wherein the carotenoid, non-oil solvent, water dispersible matrix and emulsifier are derived from natural sources

There is a need for swatches that are able to monitor lipase activity, lipid stain removal as well as methods for testing wash performance of lipases.

### SUMMARY OF THE INVENTION

In a first aspect the present invention relates to a swatch comprising a fabric and a colored lipid stain, wherein said colored lipid stain comprises one or more e.g. several lipase substrates, at least one carotenoid, and optionally a color preserving agent.

In a second aspect the present invention relates to method for preparing the swatch comprising: (a) applying a colored lipid stain to a fabric; (b) heating the swatch to a temperature above the melting temperature of the lipase substrate(s).

In a third aspect the present invention relates to use of the swatch for measuring lipase activity or for monitoring lipid stain removal.

In a fourth aspect the present invention relates to method for testing wash performance of an enzyme with lipase activity comprising: (a) contacting the swatch with a detergent composition comprising said enzyme; (b) rinsing the swatch with water; (c) drying the swatch; and (d) measuring the color intensity of the swatch at one or more wavelengths; and comparing said color intensity with the color intensity of a control swatch.

In a fifth aspect the present invention relates to a colored lipid stain comprising one or more e.g. several lipase substrates, at least one coloring agent and optionally a color preserving agent, wherein the one or more e.g. several lipase substrates constitute more than 20 wt % based on the weight of the colored lipid stain.

### DEFINITIONS

Lipase: The terms “lipase”, “lipase enzyme”, “lipolytic enzyme”, “lipid esterase”, “lipolytic polypeptide”, and “lipolytic protein” refers to an enzyme in class EC3.1.1 as defined by Enzyme Nomenclature. It may have lipase activity (triacylglycerol lipase, EC3.1.1.3), cutinase activity (EC3.1.1.74), sterol esterase activity (EC3.1.1.13) and/or wax-ester hydrolase activity (EC3.1.1.50). For purposes of the present invention, lipase activity is determined according to the procedure described in the Examples.

Lipase substrate: The term “lipase substrate” may be any substrate that is subject to the activity of a lipase as defined above.

Fabric: The term “fabric” as used herein refers to any suitable fabric, textile and/or linen serving the purpose of the present invention as described.

Wash performance: In the present context the term “wash performance” is used as an enzyme's ability to remove lipid or lipid-containing stains present on a fabric to be cleaned. The wash performance may be quantified by calculating the delta remission index value defined in the Methods section below. The wash performance may be measured on all types of suitable fabrics as described including any fabrics used in laundry, and industrial and institutional cleaning.

### DETAILED DISCLOSURE OF THE INVENTION

The present invention relates to a swatch suitable for testing lipase wash performance. The swatch is made of a piece of fabric comprising a lipid stain composition. Said lipid stain composition comprises one or more e.g. several lipase substrates and a coloring agent. The lipid stain composition may further comprise a color preserving agent.

### Fabric

In the present context a swatch is a piece of fabric such as conventional fabrics that may be used for manufacture of clothes and textile subjected to washing processes including house hold laundering as well as industrial and institutional laundering. The fabric may be any fabric made from natural plant fibres, e.g. cotton and linen; animal based fibres, e.g. wool and silk; or synthetic fibres, e.g. acrylic, polyester, polyamide, and elastane; or any combinations thereof.

The fabric may be woven or non-woven e.g. knitted. In some aspects the fabric is knitted. The fabric may be soft or stiff. In some aspects the fabric is containing cellulose. In some aspects the fabric may be woven or non-woven e.g. knitted fabric containing cellulose. In some aspects the fabric is a knitted fabric containing cellulose.

The fabric may be used directly i.e. untreated, or pre-treated. The pre-treatment may be exposure to any suitable agent that may clean the fabric and/or remove undesired material from the fabric. The undesired material may be starch and/or other impurities. In some aspects the invention relates to pre-treated fabrics wherein starch has been removed.

The fabric may be cut into any suitable size that fits the assay in which it is going to be used. Various assays known in the art is AMSA, TOM, LOM, Full Scale Wash (FSW) etc. The size of the fabric may also vary according to how many test areas should be comprised on the same piece of fabric.

### Colored Lipid Stain

The lipid stain composition comprises at least one or more e.g. several substrates, at least one coloring agent and optionally a color preserving agent. The at least one coloring agent, color preserving agent and other ingredients should optimally be dispersible in the substrate.

Substrate—

The substrate may be any substrate that can be converted by lipase. Enzymes with lipase activity are capable of hydrolysing triglycerides by releasing free fatty acids. In one embodiment the substrate is triglyceride. Triglycerides may be hydrolysed by enzymes with lipase activity into free fatty acids and glycerol. The lipase substrate may originate from animal, vegetable or mineral sources such as e.g. oil, fat, or any combination thereof. Oils includes such as coconut oil, palm oil, olive oil, sesame oil, cottonseed oil, sunflower oil, corn oil, safflower oil, canola oil, soybean oil, fish oil terpenoid and essential oils or any combination thereof. Animal fat may e.g. be lard, tallow, beef fat, milk fat, e.g. in the form of cream, butter, or free milk fat.

The substrate will constitute the main component of the lipid stain composition. The substrate is any lipid capable of dispersing the at least one coloring agent such as beta-carotene and an optionally color preserving agent such as tocopherol.

Coloring Agent—

Carotenoids are organic pigments which are split into two classes; the oxygen containing xanthophyls such as e.g. lutein and zeaxanthin, and the unoxygenated i.e. oxygen free carotenes such as e.g. alpha-carotene, beta-carotene, and lycopene. Carotenoids are usually lipophilic due to the presence of long unsaturated aliphatic chains as in some fatty acids. The most well-known carotenoid is carotene is found in carrots (and apricots) is responsible for their bright orange color. Crude palm oil, however, is the richest source of carotenoids in nature in terms of retinol (provitamin A) equivalent. Vietnamese Gac fruit contains the highest known concentration of the carotenoid lycopene. Beta-carotene is typically derived by extraction from plant sources such as algae or it may be produced synthetically. Natural and synthetic beta-carotene may be incorporated in the present invention and is readily available from several commercial sources.

The presence of beta-carotene in the lipid stain can indicate the lipid loading on the swatch. If the substrate comprises lipids which can be converted by an enzyme, the swatch may be used to assess the lipid stain removal of enzymes with lipase activity. Enzymes with lipase activity are capable of hydrolyzing and removing the lipids in the assistance of surfactants, which results in reduced color retention on the swatch.

In some aspects of the invention the lipid stain comprises a carotenoid. In some aspects of the invention the carotenoid is a xanthophyl or a carotene. In some aspects of the invention the xanthophyl is lutein or zeaxanthin. In some aspects of the invention the carotene is alpha-carotene, beta-carotene, or lycopene. Further suitable carotenoids are other fat soluble retinoids such as astaxanthin, canthaxanthin, phytoene, apo carotenal, retinol, capsanthin, zeaxanthin, beta-cryptoxanthin, phytofluene and any mixtures thereof.

In some aspects of the invention the lipid stain comprises up 10 wt %, such as e.g. 9 wt %, 8 wt %, 7 wt %, 6 wt %, 5 wt %, 4 wt %, 3 wt %, 2 wt % or 1 wt % of a coloring agent based on the weights of lipids. In some aspects of the invention the lipid stain comprises up 10 wt %, such as e.g. 9 wt %, 8 wt %, 7 wt %, 6 wt %, 5 wt %, 4 wt %, 3 wt %, 2 wt % or 1 wt % of a cartenoid based on the weights of lipids. In some aspects of the invention the lipid stain comprises up 10 wt %, such as e.g. 9 wt %, 8 wt %, 7 wt %, 6 wt %, 5 wt %, 4 wt %, 3 wt %, 2 wt % or 1 wt % of beta-carotene based on the weights of lipids. In some aspects of the invention the lipid stain comprises 0.001-0.4 wt %; 0.001-0.3 wt %; 0.001-0.2 wt %; 0.001-0.1 wt %; 0.001-0.05 wt %; 0.005-0.05 wt %; 0.01-0.05 wt %, such as 0.01 wt %; 0.02 wt %; 0.03 wt %; 0.04 wt %; or 0.05 wt % of a coloring agent based on the weight of lipids. In some aspects of the invention the lipid stain comprises 0.001-0.4 wt %; 0.001-0.3 wt %; 0.001-0.2 wt %; 0.001-0.1 wt %; 0.0001-0.05 wt %; 0.005-0.05 wt %; 0.01-0.05 wt %, such as 0.01 wt %; 0.02 wt %; 0.03 wt %; 0.04 wt %; or 0.05 wt % of a cartenoid based on the weight of lipids. In some aspects of the invention the lipid stain comprises 0.001-0.4 wt %; 0.001-0.3 wt %; 0.001-0.2 wt %; 0.001-0.1 wt %; 0.0001-0.05 wt %; 0.005-0.05 wt %; 0.01-0.05 wt %, such as 0.01 wt %; 0.02 wt %; 0.03 wt %; 0.04 wt %; or 0.05 wt % of beta-carotene based on the weight of lipids.

Color Preserving Agent—

A color preserving agent may be included in the lipid stain. This may be of particular relevance when using coloring agents that fade and/or are subject to degradation like e.g. carotenes such as beta-carotene which is degraded when subjected to air, ultraviolet light, or high temperatures. In some aspects the color preserving agent is an anti-oxidant. In some aspects the color preserving agent is tocopherols, Vitamin C, or Ascorbyl palmitate, Butylated hydroxyanisole (BHA), Butylated hydroxytoluene (BHT), Propyl gallate (PG), tert-Butylhydroquinone (TBHQ), Ascorbyl palmitate 2,4,5 Trihydroxy butyrophenone (THBP), or any combination thereof.

Tocopherols are commonly referred to as Vitamin E alcohols. The isomers which comprise Vitamin E alcohol are the alpha, beta, gamma, and delta forms. The acetate form of Vitamin E does not function as an antioxidant in food systems. Vitamin E occurs naturally in plants where it is found in wheat germ, vegetable oils, soybeans, and whole grains. Tocopherols may also be made synthetically. In some aspects the color preserving agent is tocopherol. In some aspect the tocopherol is a dl-alpha-tocopherol.

Vitamin C or L-ascorbic acid, or simply ascorbate (the anion of ascorbic acid) is an essential nutrient for humans and certain other animal species which may also act as an antioxidant.

Ascorbyl palmitate is an ester formed from ascorbic acid and palmitic acid creating a fat-soluble form of vitamin C. It is also known as an anti-oxidant food additive (E304).

In some aspects the lipid stain composition comprises 0.001-0.4 wt %, 0.005-0.3 wt %; 0.01-0.2 wt %; 0.01 wt %; 0.02 wt %; 0.03 wt %; 0.04 wt %; 0.05 wt %; 0.06 wt %; 0.07 wt %; 0.08 wt %; 0.09 wt %; 0.10 wt %; 0.15 wt %; or 0.02 wt % of a color preserving agent based on the weight of lipids. In some aspects the lipid stain composition comprises 0.001-0.4 wt %, 0.005-0.3 wt %; 0.01-0.2 wt %; 0.01 wt %; 0.02 wt %; 0.03 wt %; 0.04 wt %; 0.05 wt %; 0.06 wt %; 0.07 wt %; 0.08 wt %; 0.09 wt %; 0.10 wt %; 0.15 wt %; or 0.02 wt % of dl-alpha-tocopherol based on the weight of lipids.

In some aspects the present invention relates to a colored lipid stain comprising one or more e.g. several lipase substrates, at least one coloring agent, and optionally a color preserving agent. In some aspects the present invention relates to a colored lipid stain comprising one or more e.g. several lipase substrates, at least one coloring agent, and a color preserving agent.

In some aspects the invention relates to a colored lipid stain comprising one or more e.g. several lipase substrates, at least one coloring agent and optionally a color preserving agent, wherein the one or more e.g. several lipase substrates constitute more than 20 wt % of the colored lipid stain. The major constituent of the colored lipid stain is the one or more e.g. several lipase substrates. The amount of said substrates is more than 20 wt % but less than 100 wt % based on the weight of the colored lipid stain. In some aspects the amount of the one or more e.g. several lipase substrates constitute more than 30 wt %; more than 40 wt %; more than 50 wt %; more than 60 wt %; more than 70 wt %; more than 80 wt %; more than 85 wt %; more than 90 wt %; more than 95 wt %; more than 96 wt %; more than 97 wt %; more than 98 wt %; more than 99 wt % but less than 100 wt % based on the weight of the colored lipid stain.

In some aspects the invention relates to a colored lipid stain, wherein the at least one coloring agent and/or the optionally color preserving agent is present in concentrations from 0.001 wt % up to and including 0.4 wt % based on the weight of the colored lipid stain.

In some aspects the invention relates to a colored lipid stain, wherein the at least one coloring agent and/or the optionally color preserving agent is individually present in concentrations of 0.001-0.4 wt %, 0.005-0.3 wt %; 0.01-0.2 wt %; 0.01 wt %; 0.02 wt %; 0.03 wt %; 0.04 wt %; 0.05 wt %; 0.06 wt %; 0.07 wt %; 0.08 wt %; 0.09 wt %; 0.10 wt %; 0.15 wt %; or 0.02 wt % based on the weight of the colored lipid stain.

In some aspects the invention relates to a colored lipid stain, wherein the at least one coloring agent is a carotenoid, preferably selected from lutein, zeaxanthin, alpha-carotene, beta-carotene, lycopene, astaxanthin, canthaxanthin, phytoene, apo carotenal, retinol, capsanthin, zeaxanthin, beta-cryptoxanthin, phytofluene or any combination thereof. In some aspects the invention relates to a colored lipid stain, wherein the at least one coloring agent is a carotenoid, preferably selected from lutein, zeaxanthin, alpha-carotene, beta-carotene, lycopene, astaxanthin, canthaxanthin, phytoene, apo carotenal, retinol, capsanthin, zeaxanthin, beta-cryptoxanthin, phytofluene or any combination thereof and the color preserving agent is dl-alpha-tocopherol.

In some aspects the invention relates to a colored lipid stain, wherein the color preserving agent is an anti-oxidant, preferably selected from tocopherols e.g. dl-alpha-tocopherol, Vitamin C, Ascorbyl palmitate, or any combination thereof. In some aspects the invention relates to a colored lipid stain, wherein the color preserving agent is an anti-oxidant, preferably selected from tocopherols e.g. dl-alpha-tocopherol, Vitamin C, Ascorbyl palmitate, or any combination thereof and the at least one coloring agent is beta-carotene.

In some aspects the invention relates to a colored lipid stain, wherein the at least one coloring agent is beta-carotene and the color preserving agent is dl-alpha-tocopherol.

Method for Preparing a Colored Lipid Stain—

For preparing the colored lipid stain desired amounts of lipase substrate(s) and optional color preserving agent may be weighed into a beaker or another suitable container and melted at an appropriate temperature such as e.g. 70° C. A coloring agent is added and the mixture stirred at an appropriate temperature such as e.g. 70° C. until complete dissolution.

The temperature at which the colored lipid stain is prepared may vary depending on the nature of the lipase substrate(s). Thus the temperature should be selected above the melting temperature of the lipase substrate(s). In some aspects the temperature may be between the melting temperature of the lipase substrate(s) and up to 50° C., 55° C., 60° C., 65° C., 70° C., 75° C., 80° C., 85° C. 90° C., 95° C. or 100° C. Selection of an increased temperature within the range may help shortening the dissolution time. The temperature should be selected whereby a homogenous dissolution of the components comprised in the colored lipid stain is obtained without compromising the activity of said components such as e.g. oxidation of coloring agents like e.g. beta-carotene. The lipase substrate(s) may be melted prior to adding the coloring agent and optionally the color preserving agent. This may be an advantage if the lipase substrate has a high melting temperature. Alternatively, all components may be added and melted simultaneously.

### Preparation of the Swatch

In one aspect the present invention relates to a method for preparing a swatch comprising preparing a colored lipid stain which is applied to the fabric after which the swatch is heated to a temperature above the melting temperature of the lipase substrate(s). In one aspect the present invention relates to the method for preparing a swatch wherein the lipid stain composition comprises at least one or more e.g. several substrates, at least one coloring agent and optionally a color preserving agent.

The swatch may be prepared by applying onto the fabric a suitable amount of the desired colored lipid stain. The application may be done using any suitable method such as e.g. adding the colored lipid stain in soluble form onto the fabric; spreading a paste of the colored lipid stain onto the fabric; spraying the colored lipid stain onto the fabric; or immersion of the fabric in a solution of the colored lipid stain.

After application of the colored lipid stain to the fabric a step of heating may optionally be conducted to augment the adherence and spreading of the colored lipid stain onto the fabric. Optionally the heating temperature should be selected above the melting temperature of the lipase substrate(s). In some aspects the heating temperature may be between the melting temperature of the lipase substrate(s) and up to 50° C., 55° C., 60° C., 65° C., 70° C., 75° C., 80° C., 85° C. 90° C., 95° C. or 100° C.

During preparation and storage of the swatches the colored lipid stain should where possible be kept dark which may be done by covering with tinfoil or in any other suitable way. The need for keeping the coloring lipid stain in the dark is dependent on the sensitivity of the coloring agent to light.

During storage the swatches may be kept at low temperature such as e.g. at or below 20° C., 15° C., 10° C., 5° C. or even at or below 0° C. Preferably they may be kept in the dark. They may be vacuum packed if not used immediately or the next day.

In one aspect the present invention relates to a method for preparing a swatch comprising: (a) applying a colored lipid stain to a fabric; (b) heating the swatch to a temperature above the melting temperature of the lipase substrate(s). In one aspect the present invention relates to a method for preparing a swatch comprising applying a colored lipid stain to a fabric.

### Use of the Swatch

In some aspects the invention relates to use of the swatch for measuring lipase activity. In some aspects the invention relates to use of the swatch for monitoring lipid stain removal. In some aspects the invention relates to use of the swatch for testing wash performance of an enzyme present in a detergent composition. In some aspects the use comprise: (a) contacting the swatch with an enzyme with lipase activity; (b) rinsing with water; (c) drying of the swatch, and (d) measuring the color intensity, remission or fluorescence of the swatch at one or more wavelengths.

The drying temperature may be any suitable temperature, such as e.g. room temperature or above. Drying at an increased temperature such as e.g. 30° C., 40° C. 50° C., 60° C., 70° C., 80° C., 90° C. or 100° C. may also be applied, either as an alternative or as a supplement. The drying time is dependent on the swatch as well as on the drying temperature. Conveniently the swatches may be dried overnight preferably at room temperature.

The activity of the enzyme on the colored lipid stain on the swatch results in a change in color that may be detected as a change in color intensity, remission or fluorescence of the swatch at one or more wavelengths when compared to an appropriate control.

In some aspects for the purpose of assessing wash performance of an enzyme said enzyme will usually be in an aqueous solution when contacting with the swatch. The aqueous solution may comprise one or more e.g. several additional components typically comprised in a detergent composition as described below.

### Wash Performance Assay

Contacting the swatch with the enzyme will usually be performed by wetting the swatch with an aqueous solution of the enzyme. Wetting may be done in any appropriate way, e.g. by spraying, or immersion of the swatch with an aqueous solution of the enzyme. Contact and rinsing may be performed by washing the swatch with the aqueous solution of the enzyme in a conventional wash process, e.g. in a conventional washing machine. Contact between the swatch and the enzyme may be performed at any appropriate temperature and for any appropriate time for testing the washing performance of an enzyme. The wash temperature may be e.g. between 5-95° C., 5-80° C., 5-70° C., 5-60° C., 5-50° C., 5-40° C., 5-30° C., 5-25° C., 5-20° C., 5-15° C., or 5-10° C. The wash time may be e.g. 10-180, 10-150, 10-120, 10-90, 10-75, 10-60, 10-45, 10-30, or 10-15 minutes. Contact between the swatch and the enzyme may e.g. be performed for a time sufficient to obtain a measurable washing effect. During contact between the swatch and the enzyme mechanical treatment may or may not be applied. Mechanical treatment may be selected from e.g. shaking, rotation, or the like, to simulate the mechanical treatment in a conventional laundry machine. The swatch may after contacting with the enzyme be rinsed one or more e.g. several times e.g. 1, 2, 3, 4, 5 times as is usual in a conventional washing process.

The contact between the swatch and enzyme may e.g. be conducted a laboratory-scale multiple washing machine, e.g. Terg-O-tometer (TOM), which simulates the action of miniature domestic washing machines with controlled conditions of speed, time and temperature or a full-scale washing machine, e.g. household top-loading washing machine or front-loading washing machine.

The contact between the swatch and enzyme may e.g. be conducted in an Automated Mechanical Stress Assay (AMSA) as described in WO02/42740. With the AMSA test the wash performance of a large quantity of small volume enzyme-detergent solutions can be examined. The AMSA plate has a number of slots for test solutions and a lid firmly squeezing the textile swatch to be washed against all the slot openings. During the washing time, the plate, test solutions, textile and lid are vigorously shaken to bring the test solution in contact with the textile and apply mechanical stress.

### Color Measurement

The lipid stain removal i.e. the performance of the enzyme is measured as the difference in color of the fabric samples contacted with that specific enzyme. The difference may be assessed visually, e.g. by comparing color and intensity with a number of prepared standards, or it may be e.g. assessed by measuring reflectance or remission at one or more wavelengths, e.g. a spectrophotometer attached to a PC. Dedicated equipment for measuring color and/or reflectance may also be used for assessing the change in color and intensity. The terms “Delta remission” or “Delta remission value” are defined herein as the result of a reflectance or remission measurement at a certain wavelength which typically is 460 nm, “Delta remission” is the remission value of the washed swatch minus the remission value of the unwashed swatch. Remission or reflectance at a certain wavelength can be abbreviated with and “R” followed by the wavelength e.g. R460 for remission at 460 nm.

In some aspects of the invention the color measurement is conducted as described in the examples supra.

The color intensity of the swatch may be evaluated while the swatch is still wet, or the swatch may be dried. Since some enzyme may still be present on the swatch even after rinsing and may lead to additional breakdown of the substrate during drying, it may be preferred to perform drying at a high/increased temperature where the enzyme is inactivated and where drying is achieved within the shortest possible time, to ensure that the effect that is assayed is due to the action of the enzyme before the drying step, and not during the drying step.

### Odor Measurement

It is known that some lipases may remain in the laundry after washing and rinsing and may be active during drying. This post-wash i.e. out-of-wash effect may be desirable or undesirable since in some instances it may lead to malodor. I may thus be desirable to assay the activity of the remaining enzyme during the drying step. Accordingly in some aspects the invention relates to use of the swatch for evaluating the odor generated by the lipase. Odor generation may be conducted by sensory test methods known in the art.

In some aspects the invention relates to a method for testing post-wash performance of an enzyme with lipase activity comprising: (a) contacting the swatch with a detergent composition comprising said enzyme; (b) rinsing the swatch with water; (c) drying the swatch; and (d) measuring the odor.

The wash performance of the enzyme and the odor generation may be tested on separate swatches or on the same swatch.

### Detergents

The non-limiting list of detergent composition components illustrated hereinafter are suitable for use in the detergent compositions and methods herein may be desirably incorporated in certain embodiments of the invention, e.g. to assist or enhance cleaning performance, for treatment of the substrate to be cleaned, or to modify the aesthetics of the composition as is the case with perfumes, colorants, dyes or the like. The levels of any such components incorporated in any compositions are in addition to any materials previously recited for incorporation. The precise nature of these additional components, and levels of incorporation thereof, will depend on the physical form of the composition and the nature of the cleaning operation for which it is to be used. Although components mentioned below are categorized by general header according to a particular functionality, this is not to be construed as a limitation, as a component may comprise additional functionalities as will be appreciated by the skilled artisan.

Unless otherwise indicated the amounts in percentage is by weight of the composition (wt %). Suitable component materials include, but are not limited to, surfactants, builders, chelating agents, dye transfer inhibiting agents, dispersants, enzymes, and enzyme stabilizers, catalytic materials, bleach activators, hydrogen peroxide, sources of hydrogen peroxide, preformed peracids, polymeric dispersing agents, clay soil removal/anti-redeposition agents, brighteners, suds suppressors, dyes, hueing dyes, perfumes, perfume delivery systems, structure elasticizing agents, fabric softeners, carriers, hydrotropes, processing aids, solvents and/or pigments. In addition to the disclosure below, suitable examples of such other components and levels of use are found in U.S. Pat. No. 5,576,282, U.S. Pat. No. 6,306,812, and U.S. Pat. No. 6,326,348 hereby incorporated by reference.

Thus, in certain embodiments the invention do not contain one or more of the following adjuncts materials: surfactants, soaps, builders, chelating agents, dye transfer inhibiting agents, dispersants, additional enzymes, enzyme stabilizers, catalytic materials, bleach activators, hydrogen peroxide, sources of hydrogen peroxide, preformed peracids, polymeric dispersing agents, clay soil removal/anti-redeposition agents, brighteners, suds suppressors, dyes, perfumes, perfume delivery systems, structure elasticizing agents, fabric softeners, carriers, hydrotropes, processing aids, solvents and/or pigments. However, when one or more components are present, such one or more components may be present as detailed below:

Surfactants—

The compositions according to the present invention may comprise a surfactant or surfactant system wherein the surfactant can be selected from nonionic surfactants, anionic surfactants, cationic surfactants, ampholytic surfactants, zwitterionic surfactants, semi-polar nonionic surfactants and mixtures thereof. When present, surfactant is typically present at a level of from 0.1 to 60 wt %, from 0.2 to 40 wt %, from 0.5 to 30 wt %, from 1 to 50 wt %, from 1 to 40 wt %, from 1 to 30 wt %, from 1 to 20 wt %, from 3 to 10 wt %, from 3 to 5 wt %, from 5 to 40 wt %, from 5 to 30 wt %, from 5 to 15 wt %, from 3 to 20 wt %, from 3 to 10 wt %, from 8 to 12 wt %, from 10 to 12 wt % or from 20 to 25 wt %.

Suitable anionic detersive surfactants include sulphate and sulphonate detersive surfactants.

Suitable sulphonate detersive surfactants include alkyl benzene sulphonate, in one aspect, C₁₀₋₁₃ alkyl benzene sulphonate. Suitable alkyl benzene sulphonate (LAS) may be obtained, by sulphonating commercially available linear alkyl benzene (LAB); suitable LAB includes low 2-phenyl LAB, such as Isochem® or Petrelab®, other suitable LAB include high 2-phenyl LAB, such as Hyblene®. A suitable anionic detersive surfactant is alkyl benzene sulphonate that is obtained by DETAL catalyzed process, although other synthesis routes, such as HF, may also be suitable. In one aspect a magnesium salt of LAS is used.

Suitable sulphate detersive surfactants include alkyl sulphate, in one aspect, C₈₋₁₈ alkyl sulphate, or predominantly C₁₂ alkyl sulphate.

Another suitable sulphate detersive surfactant is alkyl alkoxylated sulphate, in one aspect, alkyl ethoxylated sulphate, in one aspect, a C₈₋₁₈ alkyl alkoxylated sulphate, in another aspect, a C₈₋₁₈ alkyl ethoxylated sulphate, typically the alkyl alkoxylated sulphate has an average degree of alkoxylation of from 0.5 to 20, or from 0.5 to 10, typically the alkyl alkoxylated sulphate is a C₈₋₁₈ alkyl ethoxylated sulphate having an average degree of ethoxylation of from 0.5 to 10, from 0.5 to 7, from 0.5 to 5 or from 0.5 to 3.

The alkyl sulphate, alkyl alkoxylated sulphate and alkyl benzene sulphonates may be linear or branched, substituted or un-substituted.

The detersive surfactant may be a mid-chain branched detersive surfactant, in one aspect, a mid-chain branched anionic detersive surfactant, in one aspect, a mid-chain branched alkyl sulphate and/or a mid-chain branched alkyl benzene sulphonate, e.g. a mid-chain branched alkyl sulphate. In one aspect, the mid-chain branches are C₁₋₄ alkyl groups, typically methyl and/or ethyl groups.

Non-limiting examples of anionic surfactants include sulfates and sulfonates, in particular, linear alkylbenzenesulfonates (LAS), isomers of LAS, branched alkylbenzenesulfonates (BABS), phenylalkanesulfonates, alpha-olefinsulfonates (AOS), olefin sulfonates, alkene sulfonates, alkane-2,3-diylbis(sulfates), hydroxyalkanesulfonates and disulfonates, alkyl sulfates (AS) such as sodium dodecyl sulfate (SDS), fatty alcohol sulfates (FAS), primary alcohol sulfates (PAS), alcohol ethersulfates (AES or AEOS or FES, also known as alcohol ethoxysulfates or fatty alcohol ether sulfates), secondary alkanesulfonates (SAS), paraffin sulfonates (PS), ester sulfonates, sulfonated fatty acid glycerol esters, alpha-sulfo fatty acid methyl esters (alpha-SFMe or SES) including methyl ester sulfonate (MES), alkyl- or alkenylsuccinic acid, dodecenyl/tetradecenyl succinic acid (DTSA), fatty acid derivatives of amino acids, diesters and monoesters of sulfo-succinic acid or soap, and combinations thereof.

Suitable non-ionic detersive surfactants are selected from the group consisting of: C₈-C₁₈ alkyl ethoxylates, such as, NEODOL®; C₆-C₁₂ alkyl phenol alkoxylates wherein the alkoxylate units may be ethyleneoxy units, propyleneoxy units or a mixture thereof; C₁₂-C₁₈ alcohol and C₆-C₁₂ alkyl phenol condensates with ethylene oxide/propylene oxide block polymers such as Pluronic®; C₁₄-C₂₂ mid-chain branched alcohols; C₁₄-C₂₂ mid-chain branched alkyl alkoxylates, typically having an average degree of alkoxylation of from 1 to 30; alkylpolysaccharides, in one aspect, alkylpolyglycosides; polyhydroxy fatty acid amides; ether capped poly(oxyalkylated) alcohol surfactants; and mixtures thereof.

Suitable non-ionic detersive surfactants include alkyl polyglucoside and/or an alkyl alkoxylated alcohol.

In one aspect, non-ionic detersive surfactants include alkyl alkoxylated alcohols, in one aspect C₈₋₁₈ alkyl alkoxylated alcohol, e.g. a C₈₋₁₈ alkyl ethoxylated alcohol, the alkyl alkoxylated alcohol may have an average degree of alkoxylation of from 1 to 50, from 1 to 30, from 1 to 20, or from 1 to 10. In one aspect, the alkyl alkoxylated alcohol may be a C₈₋₁₈ alkyl ethoxylated alcohol having an average degree of ethoxylation of from 1 to 10, from 1 to 7, more from 1 to 5 or from 3 to 7. The alkyl alkoxylated alcohol can be linear or branched, and substituted or un-substituted. Suitable nonionic surfactants include Lutensol®.

Non-limiting examples of nonionic surfactants include alcohol ethoxylates (AE or AEO), alcohol propoxylates, propoxylated fatty alcohols (PFA), alkoxylated fatty acid alkyl esters, such as ethoxylated and/or propoxylated fatty acid alkyl esters, alkylphenol ethoxylates (APE), nonylphenol ethoxylates (NPE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamides (PFAM), polyhydroxyalkyl fatty acid amides, or N-acyl N-alkyl derivatives of glucosamine (glucamides, GA, or fatty acid glucamides, FAGA), as well as products available under the trade names SPAN and TWEEN, and combinations thereof.

Suitable cationic detersive surfactants include alkyl pyridinium compounds, alkyl quaternary ammonium compounds, alkyl quaternary phosphonium compounds, alkyl ternary sulphonium compounds, and mixtures thereof.

Suitable cationic detersive surfactants are quaternary ammonium compounds having the general formula: (R)(R₁)(R₂)(R₃)N⁺ X⁻, wherein, R is a linear or branched, substituted or unsubstituted C₆₋₁₈ alkyl or alkenyl moiety, R₁ and R₂ are independently selected from methyl or ethyl moieties, R₃ is a hydroxyl, hydroxymethyl or a hydroxyethyl moiety, X is an anion which provides charge neutrality, suitable anions include: halides, e.g. chloride; sulphate; and sulphonate. Suitable cationic detersive surfactants are mono-C₆₋₁₈ alkyl mono-hydroxyethyl di-methyl quaternary ammonium chlorides. Highly suitable cationic detersive surfactants are mono-C₈₋₁₀ alkyl mono-hydroxyethyl di-methyl quaternary ammonium chloride, mono-C₁₀₋₁₂ alkyl mono-hydroxyethyl di-methyl quaternary ammonium chloride and mono-C₁₀ alkyl mono-hydroxyethyl di-methyl quaternary ammonium chloride.

Non-limiting examples of cationic surfactants include alkyldimethylethanolamine quat (ADMEAQ), cetyltrimethylammonium bromide (CTAB), dimethyldistearylammonium chloride (DSDMAC), and alkylbenzyldimethylammonium, alkyl quaternary ammonium compounds, alkoxylated quaternary ammonium (AQA) compounds, ester quats, and combinations thereof.

Suitable amphoteric/zwitterionic surfactants include amine oxides and betaines such as alkyldimethylbetaines, sulfobetaines, or combinations thereof. Amine-neutralized anionic surfactants—Anionic surfactants of the present invention and adjunct anionic cosurfactants, may exist in an acid form, and said acid form may be neutralized to form a surfactant salt which is desirable for use in the present detergent compositions. Typical agents for neutralization include the metal counterion base such as hydroxides, eg, NaOH or KOH. Further preferred agents for neutralizing anionic surfactants of the present invention and adjunct anionic surfactants or cosurfactants in their acid forms include ammonia, amines, or alkanolamines. Alkanolamines are preferred. Suitable non-limiting examples including monoethanolamine, diethanolamine, triethanolamine, and other linear or branched alkanolamines known in the art; e.g., highly preferred alkanolamines include 2-amino-1-propanol, 1-aminopropanol, monoisopropanolamine, or 1-amino-3-propanol. Amine neutralization may be done to a full or partial extent, e.g. part of the anionic surfactant mix may be neutralized with sodium or potassium and part of the anionic surfactant mix may be neutralized with amines or alkanolamines.

Non-limiting examples of semipolar surfactants include amine oxides (AO) such as alkyldimethylamineoxide

Surfactant systems comprising mixtures of one or more anionic and in addition one or more nonionic surfactants optionally with an additional surfactant such as a cationic surfactant, may be preferred. Preferred weight ratios of anionic to nonionic surfactant are at least 2:1, or at least 1:1 to 1:10.

Soap—

The compositions herein may contain soap. Without being limited by theory, it may be desirable to include soap as it acts in part as a surfactant and in part as a builder and may be useful for suppression of foam and may furthermore interact favorably with the various cationic compounds of the composition to enhance softness on textile fabrics treaded with the inventive compositions. Any soap known in the art for use in laundry detergents may be utilized. In one embodiment, the compositions contain from 0 wt % to 20 wt %, from 0.5 wt % to 20 wt %, from 4 wt % to 10 wt %, or from 4 wt % to 7 wt % of soap.

Examples of soap useful herein include oleic acid soaps, palmitic acid soaps, palm kernel fatty acid soaps, and mixtures thereof. Typical soaps are in the form of mixtures of fatty acid soaps having different chain lengths and degrees of substitution. One such mixture is topped palm kernel fatty acid.

In one embodiment, the soap is selected from free fatty acid. Suitable fatty acids are saturated and/or unsaturated and can be obtained from natural sources such a plant or animal esters (e.g., palm kernel oil, palm oil, coconut oil, babassu oil, safflower oil, tall oil, castor oil, tallow and fish oils, grease, and mixtures thereof), or synthetically prepared (e.g., via the oxidation of petroleum or by hydrogenation of carbon monoxide via the Fisher Tropsch process).

Examples of suitable saturated fatty acids for use in the compositions of this invention include captic, lauric, myristic, palmitic, stearic, arachidic and behenic acid. Suitable unsaturated fatty acid species include: palmitoleic, oleic, linoleic, linolenic and ricinoleic acid. Examples of preferred fatty acids are saturated Cn fatty acid, saturated Ci₂-Ci₄ fatty acids, and saturated or unsaturated Cn to Ci₈ fatty acids, and mixtures thereof.

When present, the weight ratio of fabric softening cationic cosurfactant to fatty acid is preferably from about 1:3 to about 3:1, more preferably from about 1:1.5 to about 1.5:1, most preferably about 1:1.

Levels of soap and of nonsoap anionic surfactants herein are percentages by weight of the detergent composition, specified on an acid form basis. However, as is commonly understood in the art, anionic surfactants and soaps are in practice neutralized using sodium, potassium or alkanolammonium bases, such as sodium hydroxide or monoethanolamine.

Hydrotropes—

The compositions of the present invention may comprise one or more hydrotropes. A hydrotrope is a compound that solubilises hydrophobic compounds in aqueous solutions (or oppositely, polar substances in a non-polar environment). Typically, hydrotropes have both hydrophilic and a hydrophobic character (so-called amphiphilic properties as known from surfactants); however the molecular structure of hydrotropes generally do not favor spontaneous self-aggregation, see e.g. review by Hodgdon and Kaler (2007), Current Opinion in Colloid & Interface Science 12: 121-128. Hydrotropes do not display a critical concentration above which self-aggregation occurs as found for surfactants and lipids forming miceller, lamellar or other well defined meso-phases. Instead, many hydrotropes show a continuous-type aggregation process where the sizes of aggregates grow as concentration increases. However, many hydrotropes alter the phase behavior, stability, and colloidal properties of systems containing substances of polar and non-polar character, including mixtures of water, oil, surfactants, and polymers. Hydrotropes are classically used across industries from pharma, personal care, food, to technical applications. Use of hydrotropes in detergent compositions allow for example more concentrated formulations of surfactants (as in the process of compacting liquid detergents by removing water) without inducing undesired phenomena such as phase separation or high viscosity.

The detergent may contain from 0 to 10 wt %, such as from 0 to 5 wt %, 0.5 to 5 wt %, or from 3% to 5 wt %, of a hydrotrope. Any hydrotrope known in the art for use in detergents may be utilized. Non-limiting examples of hydrotropes include sodium benzenesulfonate, sodium p-toluene sulfonate (STS), sodium xylene sulfonate (SXS), sodium cumene sulfonate (SCS), sodium cymene sulfonate, amine oxides, alcohols and polyglycolethers, sodium hydroxynaphthoate, sodium hydroxynaphthalene sulfonate, sodium ethylhexyl sulfate, and combinations thereof.

Builders—

The compositions of the present invention may comprise one or more builders, co-builders, builder systems or a mixture thereof. When a builder is used, the cleaning composition will typically comprise from 0 to 65 wt %, at least 1 wt %, from 2 to 60 wt % or from 5 to 10 wt % builder. In a dish wash cleaning composition, the level of builder is typically 40 to 65 wt % or 50 to 65 wt %. The composition may be substantially free of builder; substantially free means “no deliberately added” zeolite and/or phosphate. Typical zeolite builders include zeolite A, zeolite P and zeolite MAP. A typical phosphate builder is sodium tri-polyphosphate.

The builder and/or co-builder may particularly be a chelating agent that forms water-soluble complexes with Ca and Mg. Any builder and/or co-builder known in the art for use in detergents may be utilized. Non-limiting examples of builders include zeolites, diphosphates (pyrophosphates), triphosphates such as sodium triphosphate (STP or STPP), carbonates such as sodium carbonate, soluble silicates such as sodium metasilicate, layered silicates (e.g., SKS-6 from Hoechst), ethanolamines such as 2-aminoethan-1-ol (MEA), iminodiethanol (DEA) and 2,2′,2″-nitrilotriethanol (TEA), and carboxymethylinulin (CMI), and combinations thereof.

The cleaning composition may include a co-builder alone, or in combination with a builder, e.g. a zeolite builder. Non-limiting examples of co-builders include homopolymers of polyacrylates or copolymers thereof, such as poly(acrylic acid) (PAA) or copoly(acrylic acid/maleic acid) (PAA/PMA). Further non-limiting examples include citrate, chelators such as aminocarboxylates, aminopolycarboxylates and phosphonates, and alkyl- or alkenylsuccinic acid. Additional specific examples include 2,2′,2″-nitrilotriacetic acid (NTA), etheylenediaminetetraacetic acid (EDTA), diethylenetriaminepentaacetic acid (DTPA), iminodisuccinic acid (IDS), ethylenediamine-N,N′-disuccinic acid (EDDS), methylglycinediacetic acid (MGDA), glutamic acid-N,N-diacetic acid (GLDA), 1-hydroxyethane-1,1-diylbis(phosphonic acid) (HEDP), ethylenediaminetetrakis(methylene)tetrakis(phosphonic acid) (EDTMPA), diethylenetriaminepentakis(methylene)pentakis(phosphonic acid) (DTPMPA), N-(2-hydroxyethyl)iminodiacetic acid (EDG), aspartic acid-N-monoacetic acid (ASMA), aspartic acid-N,N-diacetic acid (ASDA), aspartic acid-N-monopropionic acid (ASMP), iminodisuccinic acid (IDA), N-(2-sulfomethyl) aspartic acid (SMAS), N-(2-sulfoethyl) aspartic acid (SEAS), N-(2-sulfomethyl) glutamic acid (SMGL), N-(2-sulfoethyl) glutamic acid (SEGL), N-methyliminodiacetic acid (MIDA), α-alanine-N,N-diacetic acid (α-ALDA), serine-N,N-diacetic acid (SEDA), isoserine-N,N-diacetic acid (ISDA), phenylalanine-N,N-diacetic acid (PHDA), anthranilic acid-N,N-diacetic acid (ANDA), sulfanilic acid-N, N-diacetic acid (SLDA), taurine-N, N-diacetic acid (TUDA) and sulfomethyl-N,N-diacetic acid (SMDA), N-(hydroxyethyl)-ethylidenediaminetriacetate (HEDTA), diethanolglycine (DEG), Diethylenetriamine Penta (Methylene Phosphonic acid) (DTPMP), aminotris(methylenephosphonic acid) (ATMP), and combinations and salts thereof. Further exemplary builders and/or co-builders are described in, e.g., WO09/102854, U.S. Pat. No. 5,977,053.

Chelating Agents and Crystal Growth Inhibitors—

The compositions herein may contain a chelating agent and/or a crystal growth inhibitor. Suitable molecules include copper, iron and/or manganese chelating agents and mixtures thereof. Suitable molecules include DTPA (Diethylene triamine pentaacetic acid), HEDP (Hydroxyethane diphosphonic acid), DTPMP (Diethylene triamine penta(methylene phosphonic acid)), 1,2-Dihydroxybenzene-3,5-disulfonic acid disodium salt hydrate, ethylenediamine, diethylene triamine, ethylenediaminedisuccinic acid (EDDS), N-hydroxyethylethylenediaminetri-acetic acid (HEDTA), triethylenetetraaminehexaacetic acid (TTHA), N-hydroxyethyliminodiacetic acid (HEIDA), dihydroxyethylglycine (DHEG), ethylenediaminetetrapropionic acid (EDTP), carboxymethyl inulin and 2-Phosphonobutane 1,2,4-tricarboxylic acid (Bayhibit® AM) and derivatives thereof. Typically the composition may comprise from 0.005 to 15 wt % or from 3.0 to 10 wt % chelating agent or crystal growth inhibitor.

Bleach Component—

The bleach component suitable for incorporation in the methods and compositions of the invention comprise one or a mixture of more than one bleach component. Suitable bleach components include bleaching catalysts, photobleaches, bleach activators, hydrogen peroxide, sources of hydrogen peroxide, pre-formed peracids and mixtures thereof. In general, when a bleach component is used, the compositions of the present invention may comprise from 0 to 30 wt %, from 0.00001 to 90 wt %, 0.0001 to 50 wt %, from 0.001 to 25 wt % or from 1 to 20 wt %. Examples of suitable bleach components include:

(1) Pre-formed peracids: Suitable preformed peracids include, but are not limited to, compounds selected from the group consisting of pre-formed peroxyacids or salts thereof, typically either a peroxycarboxylic acid or salt thereof, or a peroxysulphonic acid or salt thereof.

The pre-formed peroxyacid or salt thereof is preferably a peroxycarboxylic acid or salt thereof, typically having a chemical structure corresponding to the following chemical formula:

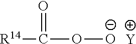

wherein: R¹⁴ is selected from alkyl, aralkyl, cycloalkyl, aryl or heterocyclic groups; the R¹⁴ group can be linear or branched, substituted or unsubstituted; and Y is any suitable counter-ion that achieves electric charge neutrality, preferably Y is selected from hydrogen, sodium or potassium. Preferably, R¹⁴ is a linear or branched, substituted or unsubstituted C₆₋₉ alkyl. Preferably, the peroxyacid or salt thereof is selected from peroxyhexanoic acid, peroxyheptanoic acid, peroxyoctanoic acid, peroxynonanoic acid, peroxydecanoic acid, any salt thereof, or any combination thereof. Particularly preferred peroxyacids are phthalimido-peroxy-alkanoic acids, in particular ε-phthahlimido peroxy hexanoic acid (PAP). Preferably, the peroxyacid or salt thereof has a melting point in the range of from 30° C. to 60° C.

The pre-formed peroxyacid or salt thereof can also be a peroxysulphonic acid or salt thereof, typically having a chemical structure corresponding to the following chemical formula:

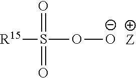

wherein: R¹⁵ is selected from alkyl, aralkyl, cycloalkyl, aryl or heterocyclic groups; the R¹⁵ group can be linear or branched, substituted or unsubstituted; and Z is any suitable counter-ion that achieves electric charge neutrality, preferably Z is selected from hydrogen, sodium or potassium. Preferably R¹⁵ is a linear or branched, substituted or unsubstituted C₆₋₉ alkyl. Preferably such bleach components may be present in the compositions of the invention in an amount from 0.01 to 50 wt % or from 0.1 to 20 wt %.

(2) Sources of hydrogen peroxide include e.g., inorganic perhydrate salts, including alkali metal salts such as sodium salts of perborate (usually mono- or tetra-hydrate), percarbonate, persulphate, perphosphate, persilicate salts and mixtures thereof. In one aspect of the invention the inorganic perhydrate salts such as those selected from the group consisting of sodium salts of perborate, percarbonate and mixtures thereof. When employed, inorganic perhydrate salts are typically present in amounts of 0.05 to 40 wt % or 1 to 30 wt % of the overall composition and are typically incorporated into such compositions as a crystalline solid that may be coated. Suitable coatings include: inorganic salts such as alkali metal silicate, carbonate or borate salts or mixtures thereof, or organic materials such as water-soluble or dispersible polymers, waxes, oils or fatty soaps. Preferably such bleach components may be present in the compositions of the invention in an amount of 0.01 to 50 wt % or 0.1 to 20 wt %.

(3) The term bleach activator is meant herein as a compound which reacts with hydrogen peroxide to form a peracid via perhydrolysis. The peracid thus formed constitutes the activated bleach. Suitable bleach activators to be used herein include those belonging to the class of esters, amides, imides or anhydrides. Suitable bleach activators are those having R—(C═O)-L wherein R is an alkyl group, optionally branched, having, when the bleach activator is hydrophobic, from 6 to 14 carbon atoms, or from 8 to 12 carbon atoms and, when the bleach activator is hydrophilic, less than 6 carbon atoms or less than 4 carbon atoms; and L is leaving group. Examples of suitable leaving groups are benzoic acid and derivatives thereof—especially benzene sulphonate. Suitable bleach activators include dodecanoyl oxybenzene sulphonate, decanoyl oxybenzene sulphonate, decanoyl oxybenzoic acid or salts thereof, 3,5,5-trimethyl hexanoyloxybenzene sulphonate, tetraacetyl ethylene diamine (TAED), sodium 4-[(3,5,5-trimethylhexanoyl)oxy]benzene-1-sulfonate (ISONOBS), 4-(dodecanoyloxy)benzene-1-sulfonate (LOBS), 4-(decanoyloxy)benzene-1-sulfonate, 4-(decanoyloxy)benzoate (DOBS or DOBA), 4-(nonanoyloxy)benzene-1-sulfonate (NOBS), and/or those disclosed in WO98/17767. A family of bleach activators is disclosed in EP624154 and particularly preferred in that family is acetyl triethyl citrate (ATC). ATC or a short chain triglyceride like triacetin has the advantage that it is environmentally friendly. Furthermore acetyl triethyl citrate and triacetin have good hydrolytical stability in the product upon storage and are efficient bleach activators. Finally ATC is multifunctional, as the citrate released in the perhydrolysis reaction may function as a builder. Alternatively, the bleaching system may comprise peroxyacids of, for example, the amide, imide, or sulfone type. The bleaching system may also comprise peracids such as 6-(phthalimido)peroxyhexanoic acid (PAP). Suitable bleach activators are also disclosed in WO98/17767. While any suitable bleach activator may be employed, in one aspect of the invention the subject cleaning composition may comprise NOBS, TAED or mixtures thereof. When present, the peracid and/or bleach activator is generally present in the composition in an amount of 0.1 to 60 wt %, 0.5 to 40 wt % or 0.6 to 10 wt % based on the fabric and home care composition. One or more hydrophobic peracids or precursors thereof may be used in combination with one or more hydrophilic peracid or precursor thereof. Preferably such bleach components may be present in the compositions of the invention in an amount of 0.01 to 50 wt %, or 0.1 to 20 wt %.

The amounts of hydrogen peroxide source and peracid or bleach activator may be selected such that the molar ratio of available oxygen (from the peroxide source) to peracid is from 1:1 to 35:1, or even 2:1 to 10:1.

(4) Diacyl peroxides—preferred diacyl peroxide bleaching species include those selected from diacyl peroxides of the general formula: R¹—C(O)—OO—(O)C—R², in which R¹ represents a C₆-C₁₈ alkyl, preferably C₆-C₁₂ alkyl group containing a linear chain of at least 5 carbon atoms and optionally containing one or more substituents (e.g. —N⁺ (CH₃)₃, —COOH or —CN) and/or one or more interrupting moieties (e.g. —CONH— or —CH═CH—) interpolated between adjacent carbon atoms of the alkyl radical, and R² represents an aliphatic group compatible with a peroxide moiety, such that R¹ and R² together contain a total of 8 to 30 carbon atoms. In one preferred aspect R¹ and R² are linear unsubstituted C₆-C₁₂ alkyl chains. Most preferably R¹ and R² are identical. Diacyl peroxides, in which both R¹ and R² are C₆-C₁₂ alkyl groups, are particularly preferred. Preferably, at least one of, most preferably only one of, the R groups (R₁ or R₂), does not contain branching or pendant rings in the alpha position, or preferably neither in the alpha nor beta positions or most preferably in none of the alpha or beta or gamma positions. In one further preferred embodiment the DAP may be asymmetric, such that preferably the hydrolysis of R1 acyl group is rapid to generate peracid, but the hydrolysis of R2 acyl group is slow.

The tetraacyl peroxide bleaching species is preferably selected from tetraacyl peroxides of the general formula: R³—C(O)—OO—C(O)—(CH₂)n-C(O)—OO—C(O)—R³, in which R³ represents a C₁-C₉ alkyl, or C₃-C₇, group and n represents an integer from 2 to 12, or 4 to 10 inclusive.

Preferably, the diacyl and/or tetraacyl peroxide bleaching species is present in an amount sufficient to provide at least 0.5 ppm, at least 10 ppm, or at least 50 ppm by weight of the wash liquor. In a preferred embodiment, the bleaching species is present in an amount sufficient to provide from 0.5 to 300 ppm, from 30 to 150 ppm by weight of the wash liquor.

Preferably the bleach component comprises a bleach catalyst (5 and 6).

(5) Preferred are organic (non-metal) bleach catalysts include bleach catalyst capable of accepting an oxygen atom from a peroxyacid and/or salt thereof, and transferring the oxygen atom to an oxidizeable substrate. Suitable bleach catalysts include, but are not limited to: iminium cations and polyions; iminium zwitterions; modified amines; modified amine oxides; N-sulphonyl imines; N-phosphonyl imines; N-acyl imines; thiadiazole dioxides; perfluoroimines; cyclic sugar ketones and mixtures thereof.

Suitable iminium cations and polyions include, but are not limited to, N-methyl-3,4-dihydroisoquinolinium tetrafluoroborate, prepared as described in Tetrahedron (1992), 49(2), 423-38 (e.g. compound 4, p. 433); N-methyl-3,4-dihydroisoquinolinium p-toluene sulphonate, prepared as described in U.S. Pat. No. 5,360,569 (e.g. Column 11, Example 1); and N-octyl-3,4-dihydroisoquinolinium p-toluene sulphonate, prepared as described in U.S. Pat. No. 5,360,568 (e.g. Column 10, Ex. 3).

Suitable iminium zwitterions include, but are not limited to, N-(3-sulfopropyl)-3,4-dihydroisoquinolinium, inner salt, prepared as described in U.S. Pat. No. 5,576,282 (e.g. Column 31, Ex. II); N-[2-(sulphooxy)dodecyl]-3,4-dihydroisoquinolinium, inner salt, prepared as described in U.S. Pat. No. 5,817,614 (e.g. Column 32, Ex. V); 2-[3-[(2-ethylhexyl)oxy]-2-(sulphooxy) propyl]-3,4-dihydroisoquinolinium, inner salt, prepared as described in WO05/047264 (e.g. p. 18, Ex. 8), and 2-[3-[(2-butyloctyl)oxy]-2-(sulphooxy)propyl]-3,4-dihydroisoquinolinium, inner salt. Suitable modified amine oxygen transfer catalysts include, but are not limited to, 1,2,3,4-tetrahydro-2-methyl-1-isoquinolinol, which can be made according to the procedures described in Tetrahedron Letters (1987), 28(48), 6061-6064. Suitable modified amine oxide oxygen transfer catalysts include, but are not limited to, sodium 1-hydroxy-N-oxy-N-[2-(sulphooxy)decyl]-1,2,3,4-tetrahydroisoquinoline.

Suitable N-sulphonyl imine oxygen transfer catalysts include, but are not limited to, 3-methyl-1,2-benzisothiazole 1,1-dioxide, prepared according to the procedure described in the Journal of Organic Chemistry (1990), 55(4), 1254-61.

Suitable N-phosphonyl imine oxygen transfer catalysts include, but are not limited to, [R-(E)]-N-[(2-chloro-5-nitrophenyl)methylene]-P-phenyl-P-(2,4,6-trimethylphenyl)-phosphinic amide, which can be made according to the procedures described in the Journal of the Chemical Society, Chemical Communications (1994), (22), 2569-70.

Suitable N-acyl imine oxygen transfer catalysts include, but are not limited to, [N(E)]-N-(phenylmethylene)acetamide, which can be made according to the procedures described in Polish Journal of Chemistry (2003), 77(5), 577-590.

Suitable thiadiazole dioxide oxygen transfer catalysts include but are not limited to, 3-methyl-4-phenyl-1,2,5-thiadiazole 1,1-dioxide, which can be made according to the procedures described in U.S. Pat. No. 5,753,599 (Column 9, Ex. 2).

Suitable perfluoroimine oxygen transfer catalysts include, but are not limited to, (Z)-2,2,3,3,4,4,4-heptafluoro-N-(nonafluorobutyl)butanimidoyl fluoride, which can be made according to the procedures described in Tetrahedron Letters (1994), 35(34), 6329-30.

Suitable cyclic sugar ketone oxygen transfer catalysts include, but are not limited to, 1,2:4,5-di-O-isopropylidene-D-erythro-2,3-hexodiuro-2,6-pyranose as prepared in U.S. Pat. No. 6,649,085 (Column 12, Ex. 1).

Preferably, the bleach catalyst comprises an iminium and/or carbonyl functional group and is typically capable of forming an oxaziridinium and/or dioxirane functional group upon acceptance of an oxygen atom, especially upon acceptance of an oxygen atom from a peroxyacid and/or salt thereof. Preferably, the bleach catalyst comprises an oxaziridinium functional group and/or is capable of forming an oxaziridinium functional group upon acceptance of an oxygen atom, especially upon acceptance of an oxygen atom from a peroxyacid and/or salt thereof. Preferably, the bleach catalyst comprises a cyclic iminium functional group, preferably wherein the cyclic moiety has a ring size of from five to eight atoms (including the nitrogen atom), preferably six atoms. Preferably, the bleach catalyst comprises an aryliminium functional group, preferably a bi-cyclic aryliminium functional group, preferably a 3,4-dihydroisoquinolinium functional group. Typically, the imine functional group is a quaternary imine functional group and is typically capable of forming a quaternary oxaziridinium functional group upon acceptance of an oxygen atom, especially upon acceptance of an oxygen atom from a peroxyacid and/or salt thereof. In another aspect, the detergent composition comprises a bleach component having a log P_{o/w} no greater than 0, no greater than −0.5, no greater than −1.0, no greater than −1.5, no greater than −2.0, no greater than −2.5, no greater than −3.0, or no greater than −3.5. The method for determining log P_{o/w} is described in more detail below.

Typically, the bleach ingredient is capable of generating a bleaching species having a X_{SO} of from 0.01 to 0.30, from 0.05 to 0.25, or from 0.10 to 0.20. The method for determining X_{SO} is described in more detail below. For example, bleaching ingredients having an isoquinolinium structure are capable of generating a bleaching species that has an oxaziridinium structure. In this example, the X_{SO} is that of the oxaziridinium bleaching species.

Preferably, the bleach catalyst has a chemical structure corresponding to the following chemical formula:

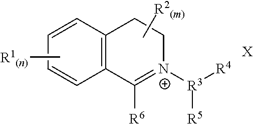

wherein: n and m are independently from 0 to 4, preferably n and m are both 0; each R¹ is independently selected from a substituted or unsubstituted radical selected from the group consisting of hydrogen, alkyl, cycloalkyl, aryl, fused aryl, heterocyclic ring, fused heterocyclic ring, nitro, halo, cyano, sulphonato, alkoxy, keto, carboxylic, and carboalkoxy radicals; and any two vicinal R¹ substituents may combine to form a fused aryl, fused carbocyclic or fused heterocyclic ring; each R² is independently selected from a substituted or unsubstituted radical independently selected from the group consisting of hydrogen, hydroxy, alkyl, cycloalkyl, alkaryl, aryl, aralkyl, alkylenes, heterocyclic ring, alkoxys, arylcarbonyl groups, carboxyalkyl groups and amide groups; any R² may be joined together with any other of R² to form part of a common ring; any geminal R² may combine to form a carbonyl; and any two R² may combine to form a substituted or unsubstituted fused unsaturated moiety; R³ is a C₁ to C₂₀ substituted or unsubstituted alkyl; R⁴ is hydrogen or the moiety Qₜ-A, wherein: Q is a branched or unbranched alkylene, t=0 or 1 and A is an anionic group selected from the group consisting of OSO₃⁻, SO₃⁻, CO₂⁻, OCO₂⁻, OPO₃²⁻, OPO₃H⁻ and OPO₂⁻; R⁵ is hydrogen or the moiety —CR¹¹R¹²—Y-G_{b}-Y_{c}—[(CR⁹R¹⁰)_{y}—O]ₖ−R⁸, wherein: each Y is independently selected from the group consisting of O, S, N—H, or N—R⁸; and each R⁸ is independently selected from the group consisting of alkyl, aryl and heteroaryl, said moieties being substituted or unsubstituted, and whether substituted or unsubstituted said moieties having less than 21 carbons; each G is independently selected from the group consisting of CO, SO₂, SO, PO and PO₂; R⁹ and R¹⁰ are independently selected from the group consisting of H and C₁-C₄ alkyl; R¹¹ and R¹² are independently selected from the group consisting of H and alkyl, or when taken together may join to form a carbonyl; b=0 or 1; c can=0 or 1, but c must=0 if b=0; y is an integer from 1 to 6; k is an integer from 0 to 20; R⁶ is H, or an alkyl, aryl or heteroaryl moiety; said moieties being substituted or unsubstituted; and X, if present, is a suitable charge balancing counterion, preferably X is present when R⁴ is hydrogen, suitable X, include but are not limited to: chloride, bromide, sulphate, methosulphate, sulphonate, p-toluenesulphonate, borontetraflouride and phosphate.

In one embodiment of the present invention, the bleach catalyst has a structure corresponding to general formula below:

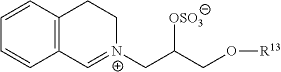

wherein R¹³ is a branched alkyl group containing from three to 24 carbon atoms (including the branching carbon atoms) or a linear alkyl group containing from one to 24 carbon atoms; preferably R¹³ is a branched alkyl group containing from eight to 18 carbon atoms or linear alkyl group containing from eight to eighteen carbon atoms; preferably R¹³ is selected from the group consisting of 2-propylheptyl, 2-butyloctyl, 2-pentylnonyl, 2-hexyldecyl, n-dodecyl, n-tetradecyl, n-hexadecyl, n-octadecyl, iso-nonyl, iso-decyl, iso-tridecyl and iso-pentadecyl; preferably R¹³ is selected from the group consisting of 2-butyloctyl, 2-pentylnonyl, 2-hexyldecyl, iso-tridecyl and iso-pentadecyl.

Preferably the bleach component comprises a source of peracid in addition to bleach catalyst, particularly organic bleach catalyst. The source of peracid may be selected from (a) pre-formed peracid; (b) percarbonate, perborate or persulfate salt (hydrogen peroxide source) preferably in combination with a bleach activator; and (c) perhydrolase enzyme and an ester for forming peracid in situ in the presence of water in a textile or hard surface treatment step.

When present, the peracid and/or bleach activator is generally present in the composition in an amount of from 0.1 to 60 wt %, from 0.5 to 40 wt % or from 0.6 to 10 wt % based on the composition. One or more hydrophobic peracids or precursors thereof may be used in combination with one or more hydrophilic peracid or precursor thereof.

The amounts of hydrogen peroxide source and peracid or bleach activator may be selected such that the molar ratio of available oxygen (from the peroxide source) to peracid is from 1:1 to 35:1, or 2:1 to 10:1.

(6) Metal-containing Bleach Catalysts—The bleach component may be provided by a catalytic metal complex. One type of metal-containing bleach catalyst is a catalyst system comprising a transition metal cation of defined bleach catalytic activity, such as copper, iron, titanium, ruthenium, tungsten, molybdenum, or manganese cations, an auxiliary metal cation having little or no bleach catalytic activity, such as zinc or aluminum cations, and a sequestrate having defined stability constants for the catalytic and auxiliary metal cations, particularly ethylenediaminetetraacetic acid, ethylenediaminetetra(methylenephosphonic acid) and water-soluble salts thereof. Such catalysts are disclosed in U.S. Pat. No. 4,430,243. Preferred catalysts are described in WO09/839406, U.S. Pat. No. 6,218,351 and WO00/012667. Particularly preferred are transition metal catalyst or ligands therefore that are cross-bridged polydentate N-donor ligands.

If desired, the compositions herein can be catalyzed by means of a manganese compound. Such compounds and levels of use are well known in the art and include, e.g., the manganese-based catalysts disclosed in U.S. Pat. No. 5,576,282.

Cobalt bleach catalysts useful herein are known, and are described e.g. in U.S. Pat. No. 5,597,936; U.S. Pat. No. 5,595,967. Such cobalt catalysts are readily prepared by known procedures, such as taught e.g. in U.S. Pat. No. 5,597,936 and U.S. Pat. No. 5,595,967.

Compositions herein may also suitably include a transition metal complex of ligands such as bispidones (U.S. Pat. No. 7,501,389) and/or macropolycyclic rigid ligands—abbreviated as “MRLs”. As a practical matter, and not by way of limitation, the compositions and processes herein can be adjusted to provide on the order of at least one part per hundred million of the active MRL species in the aqueous washing medium, and will typically provide from 0.005 to 25 ppm, from 0.05 to 10 ppm, or from 0.1 to 5 ppm, of the MRL in the wash liquor.

Suitable transition-metals in the instant transition-metal bleach catalyst include e.g. manganese, iron and chromium. Suitable MRLs include 5,12-diethyl-1,5,8,12-tetraazabicyclo[6.6.2]hexadecane. Suitable transition metal MRLs are readily prepared by known procedures, such as taught e.g. in U.S. Pat. No. 6,225,464 and WO00/32601.

(7) Photobleaches—suitable photobleaches include e.g. sulfonated zinc phthalocyanine sulfonated aluminium phthalocyanines, xanthene dyes and mixtures thereof. Preferred bleach components for use in the present compositions of the invention comprise a hydrogen peroxide source, bleach activator and/or organic peroxyacid, optionally generated in situ by the reaction of a hydrogen peroxide source and bleach activator, in combination with a bleach catalyst. Preferred bleach components comprise bleach catalysts, preferably organic bleach catalysts, as described above.

Particularly preferred bleach components are the bleach catalysts in particular the organic bleach catalysts.

Exemplary bleaching systems are also described, e.g. in WO2007/087258, WO2007/087244, WO2007/087259 and WO2007/087242.

Fabric Hueing Agents—

The composition may comprise a fabric hueing agent. Suitable fabric hueing agents include dyes, dye-clay conjugates, and pigments. Suitable dyes include small molecule dyes and polymeric dyes. Suitable small molecule dyes include small molecule dyes selected from the group consisting of dyes falling into the Color Index (C.I.) classifications of Direct Blue, Direct Red, Direct Violet, Acid Blue, Acid Red, Acid Violet, Basic Blue, Basic Violet and Basic Red, or mixtures thereof.

In another aspect, suitable small molecule dyes include small molecule dyes selected from the group consisting of Color Index (Society of Dyers and Colorists, Bradford, UK) numbers Direct Violet 9, Direct Violet 35, Direct Violet 48, Direct Violet 51, Direct Violet 66, Direct Violet 99, Direct Blue 1, Direct Blue 71, Direct Blue 80, Direct Blue 279, Acid Red 17, Acid Red 73, Acid Red 88, Acid Red 150, Acid Violet 15, Acid Violet 17, Acid Violet 24, Acid Violet 43, Acid Red 52, Acid Violet 49, Acid Violet 50, Acid Blue 15, Acid Blue 17, Acid Blue 25, Acid Blue 29, Acid Blue 40, Acid Blue 45, Acid Blue 75, Acid Blue 80, Acid Blue 83, Acid Blue 90 and Acid Blue 113, Acid Black 1, Basic Violet 1, Basic Violet 3, Basic Violet 4, Basic Violet 10, Basic Violet 35, Basic Blue 3, Basic Blue 16, Basic Blue 22, Basic Blue 47, Basic Blue 66, Basic Blue 75, Basic Blue 159 and mixtures thereof. In another aspect, suitable small molecule dyes include small molecule dyes selected from the group consisting of Color Index (Society of Dyers and Colorists, Bradford, UK) numbers Acid Violet 17, Acid Violet 43, Acid Red 52, Acid Red 73, Acid Red 88, Acid Red 150, Acid Blue 25, Acid Blue 29, Acid Blue 45, Acid Blue 113, Acid Black 1, Direct Blue 1, Direct Blue 71, Direct Violet 51 and mixtures thereof. In another aspect, suitable small molecule dyes include small molecule dyes selected from the group consisting of Color Index (Society of Dyers and Colorists, Bradford, UK) numbers Acid Violet 17, Direct Blue 71, Direct Violet 51, Direct Blue 1, Acid Red 88, Acid Red 150, Acid Blue 29, Acid Blue 113 or mixtures thereof.

Suitable polymeric dyes include polymeric dyes selected from the group consisting of polymers containing conjugated chromogens (dye-polymer conjugates) and polymers with chromogens co-polymerized into the backbone of the polymer and mixtures thereof.

In another aspect, suitable polymeric dyes include polymeric dyes selected from the group consisting of fabric-substantive colorants sold under the name of Liquitint® (Milliken), dye-polymer conjugates formed from at least one reactive dye and a polymer selected from the group consisting of polymers comprising a moiety selected from the group consisting of a hydroxyl moiety, a primary amine moiety, a secondary amine moiety, a thiol moiety and mixtures thereof. In still another aspect, suitable polymeric dyes include polymeric dyes selected from the group consisting of Liquitint® Violet CT, carboxymethyl cellulose (CMC) conjugated with a reactive blue, reactive violet or reactive red dye such as CMC conjugated with C.I. Reactive Blue 19, sold by Megazyme, Wicklow, Ireland under the product name AZO-CM-CELLULOSE, product code S-ACMC, alkoxylated triphenyl-methane polymeric colorants, alkoxylated thiophene polymeric colorants, and mixtures thereof.

Preferred hueing dyes include the whitening agents found in WO08/87497. These whitening agents may be characterized by the following structure (I):

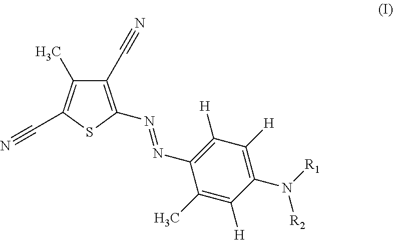

wherein R₁ and R₂ can independently be selected from:
a) [(CH₂CR′HO)ₓ(CH₂CR″HO)_{y}H]
   wherein R′ is selected from the group consisting of H, CH₃, CH₂O(CH₂CH₂O)_{z}H, and mixtures thereof; wherein R″ is selected from the group consisting of H, CH₂O(CH₂CH₂O)_{z}H, and mixtures thereof; wherein x+y≦5; wherein y≧1; and wherein z=0 to 5;
b) R₁=alkyl, aryl or aryl alkyl and R₂=[(CH₂CR′HO)ₓ(CH₂CR″HO)_{y}H]
   wherein R′ is selected from the group consisting of H, CH₃, CH₂O(CH₂CH₂O)_{z}H, and mixtures thereof; wherein R″ is selected from the group consisting of H, CH₂O(CH₂CH₂O)_{z}H, and mixtures thereof; wherein x+y≦10; wherein y≧1; and wherein z=0 to 5;
c) R₁=[CH₂CH₂(OR₃)CH₂OR₄] and R₂=[CH₂CH₂(O R₃)CH₂O R₄]
   wherein R₃ is selected from the group consisting of H, (CH₂CH₂O)_{z}H, and mixtures thereof; and
   wherein z=0 to 10;
   wherein R₄ is selected from the group consisting of (C₁-C₁₆)alkyl, aryl groups, and mixtures thereof; and
   d) wherein R1 and R2 can independently be selected from the amino addition product of styrene oxide, glycidyl methyl ether, isobutyl glycidyl ether, isopropylglycidyl ether, t-butyl glycidyl ether, 2-ethylhexylgycidyl ether, and glycidylhexadecyl ether, followed by the addition of from 1 to 10 alkylene oxide units.

A preferred whitening agent of the present invention may be characterized by the following structure (II):

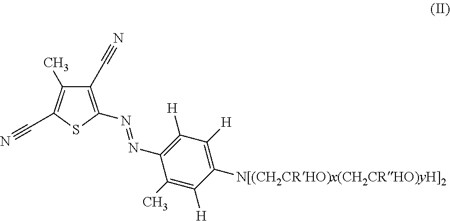

wherein R′ is selected from the group consisting of H, CH₃, CH₂O(CH₂CH₂O)_{z}H, and mixtures thereof; wherein R″ is selected from the group consisting of H, CH₂O(CH₂CH₂O)_{z}H, and mixtures thereof; wherein x+y≦5; wherein y≧1; and wherein z=0 to 5.

A further preferred whitening agent of the present invention may be characterized by the following structure (III):

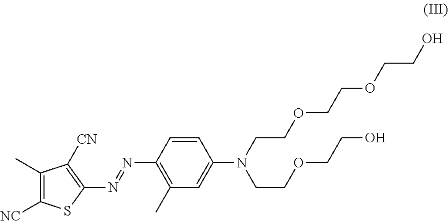 [SMILES: Cc1cc(N(CCOCCO)CCOCCOCCO)ccc1/N=N/c1sc(C#N)c(C)c1C#N]

typically comprising a mixture having a total of 5 EO groups. Suitable preferred molecules are those in Structure I having the following pendant groups in “part a” above.

TABLE A

| | |
|---|---|
| R1 | R2 |

| | R′ | R″ | x | y | R′ | R″ | x | y |
|---|---|---|---|---|---|---|---|---|
| a | H | H | 3 | 1 | H | H | 0 | 1 |
| b | H | H | 2 | 1 | H | H | 1 | 1 |
| c = b | H | H | 1 | 1 | H | H | 2 | 1 |
| d = a | H | H | 0 | 1 | H | H | 3 | 1 |

Further whitening agents of use include those described in US2008/34511 (Unilever). A preferred agent is “Violet 13”.

Suitable dye clay conjugates include dye clay conjugates selected from the group comprising at least one cationic/basic dye and a smectite clay, and mixtures thereof. In another aspect, suitable dye clay conjugates include dye clay conjugates selected from the group consisting of one cationic/basic dye selected from the group consisting of C.I. Basic Yellow 1 through 108, C.I. Basic Orange 1 through 69, C.I. Basic Red 1 through 118, C.I. Basic Violet 1 through 51, C.I. Basic Blue 1 through 164, C.I. Basic Green 1 through 14, C.I. Basic Brown 1 through 23, CI Basic Black 1 through 11, and a clay selected from the group consisting of Montmorillonite clay, Hectorite clay, Saponite clay and mixtures thereof. In still another aspect, suitable dye clay conjugates include dye clay conjugates selected from the group consisting of: Montmorillonite Basic Blue B7 C.I. 42595 conjugate, Montmorillonite Basic Blue B9 C.I. 52015 conjugate, Montmorillonite Basic Violet V3 C.I. 42555 conjugate, Montmorillonite Basic Green G1 C.I. 42040 conjugate, Montmorillonite Basic Red R1 C.I. 45160 conjugate, Montmorillonite C.I. Basic Black 2 conjugate, Hectorite Basic Blue B7 C.I. 42595 conjugate, Hectorite Basic Blue B9 C.I. 52015 conjugate, Hectorite Basic Violet V3 C.I. 42555 conjugate, Hectorite Basic Green G1 C.I. 42040 conjugate, Hectorite Basic Red R1 C.I. 45160 conjugate, Hectorite C.I. Basic Black 2 conjugate, Saponite Basic Blue B7 C.I. 42595 conjugate, Saponite Basic Blue B9 C.I. 52015 conjugate, Saponite Basic Violet V3 C.I. 42555 conjugate, Saponite Basic Green G1 C.I. 42040 conjugate, Saponite Basic Red R1 C.I. 45160 conjugate, Saponite C.I. Basic Black 2 conjugate and mixtures thereof.

Suitable pigments include pigments selected from the group consisting of flavanthrone, indanthrone, chlorinated indanthrone containing from 1 to 4 chlorine atoms, pyranthrone, dichloropyranthrone, monobromodichloropyranthrone, dibromodichloropyranthrone, tetrabromopyranthrone, perylene-3,4,9,10-tetracarboxylic acid diimide, wherein the imide groups may be unsubstituted or substituted by C1-C3-alkyl or a phenyl or heterocyclic radical, and wherein the phenyl and heterocyclic radicals may additionally carry substituents which do not confer solubility in water, anthrapyrimidinecarboxylic acid amides, violanthrone, isoviolanthrone, dioxazine pigments, copper phthalocyanine which may contain up to 2 chlorine atoms per molecule, polychloro-copper phthalocyanine or polybromochloro-copper phthalocyanine containing up to 14 bromine atoms per molecule and mixtures thereof. In another aspect, suitable pigments include pigments selected from the group consisting of Ultramarine Blue (CA. Pigment Blue 29), Ultramarine Violet (CA. Pigment Violet 15) and mixtures thereof.

The aforementioned fabric hueing agents can be used in combination (any mixture of fabric hueing agents can be used). Suitable hueing agents are described in more detail in U.S. Pat. No. 7,208,459. Preferred levels of dye in compositions of the invention are 0.00001 to 0.5 wt %, or 0.0001 to 0.25 wt %. The concentration of dyes preferred in water for the treatment and/or cleaning step is from 1 ppb to 5 ppm, 10 ppb to 5 ppm or 20 ppb to 5 ppm. In preferred compositions, the concentration of surfactant will be from 0.2 to 3 g/I.

Encapsulates—

The composition may comprise an encapsulate. In one aspect, an encapsulate comprising a core, a shell having an inner and outer surface, said shell encapsulating said core.

In one aspect of said encapsulate, said core may comprise a material selected from the group consisting of perfumes; brighteners; dyes; insect repellants; silicones; waxes; flavors; vitamins; fabric softening agents; skin care agents in one aspect, paraffins; enzymes; anti-bacterial agents; bleaches; sensates; and mixtures thereof; and said shell may comprise a material selected from the group consisting of polyethylenes; polyamides; polyvinylalcohols, optionally containing other co-monomers; polystyrenes; polyisoprenes; polycarbonates; polyesters; polyacrylates; aminoplasts, in one aspect said aminoplast may comprise a polyureas, polyurethane, and/or polyureaurethane, in one aspect said polyurea may comprise polyoxymethyleneurea and/or melamine formaldehyde; polyolefins; polysaccharides, in one aspect said polysaccharide may comprise alginate and/or chitosan; gelatin; shellac; epoxy resins; vinyl polymers; water insoluble inorganics; silicone; and mixtures thereof.

In one aspect of said encapsulate, said core may comprise perfume.

In one aspect of said encapsulate, said shell may comprise melamine formaldehyde and/or cross linked melamine formaldehyde.

In a one aspect, suitable encapsulates may comprise a core material and a shell, said shell at least partially surrounding said core material, is disclosed. 85% or 90% of said encapsulates may have a fracture strength of from 0.2 to 10 MPa, from 0.4 to 5 MPa, from 0.6 to 3.5 MPa, or from 0.7 to 3 MPa; and a benefit agent leakage of from 0 to 30%, from 0 to 20%, or from 0 to 5%.

In one aspect, 85% or 90% of said encapsulates may have a particle size from 1 to 80 microns, from 5 to 60 microns, from 10 to 50 microns, or from 15 to 40 microns.

In one aspect, 85% or 90% of said encapsulates may have a particle wall thickness from 30 to 250 nm, from 80 to 180 nm, or from 100 to 160 nm.

In one aspect, said encapsulates' core material may comprise a material selected from the group consisting of a perfume raw material and/or optionally a material selected from the group consisting of vegetable oil, including neat and/or blended vegetable oils including castor oil, coconut oil, cottonseed oil, grape oil, rapeseed, soybean oil, corn oil, palm oil, linseed oil, safflower oil, olive oil, peanut oil, coconut oil, palm kernel oil, castor oil, lemon oil and mixtures thereof; esters of vegetable oils, esters, including dibutyl adipate, dibutyl phthalate, butyl benzyl adipate, benzyl octyl adipate, tricresyl phosphate, trioctyl phosphate and mixtures thereof; straight or branched chain hydrocarbons, including those straight or branched chain hydrocarbons having a boiling point of greater than about 80° C.; partially hydrogenated terphenyls, dialkyl phthalates, alkyl biphenyls, including monoisopropylbiphenyl, alkylated naphthalene, including dipropylnaphthalene, petroleum spirits, including kerosene, mineral oil and mixtures thereof; aromatic solvents, including benzene, toluene and mixtures thereof; silicone oils; and mixtures thereof.

In one aspect, said encapsulates' wall material may comprise a suitable resin including the reaction product of an aldehyde and an amine, suitable aldehydes include, formaldehyde. Suitable amines include melamine, urea, benzoguanamine, glycoluril, and mixtures thereof. Suitable melamines include methylol melamine, methylated methylol melamine, imino melamine and mixtures thereof. Suitable ureas include dimethylol urea, methylated dimethylol urea, urea-resorcinol, and mixtures thereof.

In one aspect, suitable formaldehyde scavengers may be employed with the encapsulates e.g. in a capsule slurry and/or added to a composition before, during or after the encapsulates are added to such composition. Suitable capsules may be made by the following teaching of US2008/0305982; and/or US2009/0247449.

In a preferred aspect the composition can also comprise a deposition aid, preferably consisting of the group comprising cationic or nonionic polymers. Suitable polymers include cationic starches, cationic hydroxyethylcellulose, polyvinylformaldehyde, locust bean gum, mannans, xyloglucans, tamarind gum, polyethyleneterephthalate and polymers containing dimethylaminoethyl methacrylate, optionally with one or monomers selected from the group comprising acrylic acid and acrylamide.

Perfumes—

In one aspect the composition comprises a perfume that comprises one or more perfume raw materials selected from the group consisting of 1,1′-oxybis-2-propanol; 1,4-cyclohexanedicarboxylic acid, diethyl ester; (ethoxymethoxy)cyclododecane; 1,3-nonanediol, monoacetate; (3-methylbutoxy)acetic acid, 2-propenyl ester; beta-methyl cyclododecaneethanol; 2-methyl-3-[(1,7,7-trimethylbicyclo[2.2.1]hept-2-yl)oxy]-1-propanol; oxacyclohexadecan-2-one; alpha-methyl-benzenemethanol acetate; trans-3-ethoxy-1,1,5-trimethylcyclohexane; 4-(1,1-dimethylethyl)cyclohexanol acetate; dodecahydro-3a,6,6,9a-tetramethylnaphtho[2,1-b]furan; beta-methyl benzenepropanal; beta-methyl-3-(1-methylethyl)benzenepropanal; 4-phenyl-2-butanone; 2-methylbutanoic acid, ethyl ester; benzaldehyde; 2-methylbutanoic acid, 1-methylethyl ester; dihydro-5-pentyl-2(3H)furanone; (2E)-1-(2,6,6-trimethyl-2-cyclohexen-1-yl)-2-buten-1-one; dodecanal; undecanal; 2-ethyl-alpha, alpha-dimethylbenzenepropanal; decanal; alpha, alpha-dimethylbenzeneethanol acetate; 2-(phenylmethylene)octanal; 2-[[3-[4-(1,1-dimethylethyl)phenyl]-2-methylpropylidene]amino]benzoic acid, methyl ester; 1-(2,6,6-trimethyl-3-cyclohexen-1-yl)-2-buten-1-one; 2-pentylcyclopentanone; 3-oxo-2-pentyl cyclopentaneacetic acid, methyl ester; 4-hydroxy-3-methoxybenzaldehyde; 3-ethoxy-4-hydroxybenzaldehyde; 2-heptylcyclopentanone; 1-(4-methylphenyl)ethanone; (3E)-4-(2,6,6-trimethyl-1-cyclohexen-1-yl)-3-buten-2-one; (3E)-4-(2,6,6-trimethyl-2-cyclohexen-1-yl)-3-buten-2-one; benzeneethanol; 2H-1-benzopyran-2-one; 4-methoxybenzaldehyde; 10-undecenal; propanoic acid, phenylmethyl ester; beta-methylbenzenepentanol; 1,1-diethoxy-3,7-dimethyl-2,6-octadiene; alpha, alpha-dimethylbenzeneethanol; (2E)-1-(2,6,6-trimethyl-1-cyclohexen-1-yl)-2-buten-1-one; acetic acid, phenylmethyl ester; cyclohexanepropanoic acid, 2-propenyl ester; hexanoic acid, 2-propenyl ester; 1,2-dimethoxy-4-(2-propenyl)benzene; 1,5-dimethyl-bicyclo[3.2.1]octan-8-one oxime; 4-(4-hydroxy-4-methylpentyl)-3-cyclohexene-1-carboxaldehyde; 3-buten-2-ol; 2-[[[2,4(or 3,5)-dimethyl-3-cyclohexen-1-yl]methylene]amino]benzoic acid, methyl ester; 8-cyclohexadecen-1-one; methyl ionone; 2,6-dimethyl-7-octen-2-ol; 2-methoxy-4-(2-propenyl) phenol; (2E)-3,7-dimethyl-2,6-Octadien-1-ol; 2-hydroxy-Benzoic acid, (3Z)-3-hexenyl ester; 2-tridecenenitrile; 4-(2,2-dimethyl-6-methylenecyclohexyl)-3-methyl-3-buten-2-one; tetrahydro-4-methyl-2-(2-methyl-1-propenyl)-2H-pyran; Acetic acid, (2-methylbutoxy)-, 2-propenyl ester; Benzoic acid, 2-hydroxy-, 3-methyl butyl ester; 2-Buten-1-one, 1-(2,6,6-tri methyl-1-cyclohexen-1-yl)-, (Z)-; Cyclopentanecarboxylic acid, 2-hexyl-3-oxo-, methyl ester; Benzenepropanal, 4-ethyl-.alpha., .alpha.-dimethyl-; 3-Cyclohexene-1-carboxaldehyde, 3-(4-hydroxy-4-methylpentyl)-; Ethanone, 1-(2,3,4,7,8,8a-hexahydro-3,6,8,8-tetramethyl-1H-3a,7-methanoazulen-5-yl)-, [3R-(3.alpha.,3a.beta.,7.beta.,8a.alpha.)]-; Undecanal, 2-methyl-2H-Pyran-2-one, 6-butyltetrahydro-; Benzenepropanal, 4-(1,1-dimethylethyl)-.alpha.-methyl-; 2(3H)-Furanone, 5-heptyldihydro-; Benzoic acid, 2-[(7-hydroxy-3,7-dimethyloctylidene)amino]-, methyl; Benzoic acid, 2-hydroxy-, phenylmethyl ester; Naphthalene, 2-methoxy-; 2-Cyclopenten-1-one, 2-hexyl-; 2(3H)-Furanone, 5-hexyldihydro-; Oxiranecarboxylic acid, 3-methyl-3-phenyl-, ethyl ester; 2-Oxabicyclo[2.2.2]octane, 1,3,3-trimethyl-; Benzenepentanol, .gamma.-methyl-; 3-Octanol, 3,7-dimethyl-; 3,7-dimethyl-2,6-octadienenitrile; 3,7-dimethyl-6-octen-1-ol; Terpineol acetate; 2-methyl-6-methylene-7-Octen-2-ol, dihydro derivative; 3a,4,5,6,7,7a-hexahydro-4,7-Methano-1H-inden-6-ol propanoate; 3-methyl-2-buten-1-ol acetate; (Z)-3-Hexen-1-ol acetate; 2-ethyl-4-(2,2,3-trimethyl-3-cyclopenten-1-yl)-2-buten-1-ol; 4-(octahydro-4,7-methano-5H-inden-5-ylidene)-butanal; 3-2,4-dimethyl-cyclohexene-1-carboxaldehyde; 1-(1,2,3,4,5,6,7,8-octahydro-2,3,8,8-tetramethyl-2-naphthalenyl)-ethanone; 2-hydroxy-benzoic acid, methyl ester; 2-hydroxy-benzoic acid, hexyl ester; 2-phenoxy-ethanol; 2-hydroxy-benzoic acid, pentyl ester; 2,3-heptanedione; 2-hexen-1-ol; 6-Octen-2-ol, 2,6-dimethyl-; damascone (alpha, beta, gamma or delta or mixtures thereof), 4,7-Methano-1H-inden-6-ol, 3a,4,5,6,7,7a-hexahydro-, acetate; 9-Undecenal; 8-Undecenal; Isocyclocitral; Ethanone, 1-(1,2,3,5,6,7,8,8a-octahydro-2,3,8,8-tetramethyl-2-naphthalenyl)-; 3-Cyclohexene-1-carboxaldehyde, 3,5-dimethyl-; 3-Cyclohexene-1-carboxaldehyde, 2,4-dimethyl-; 1,6-Octadien-3-ol, 3,7-dimethyl-; 1,6-Octadien-3-ol, 3,7-dimethyl-, acetate; Lilial (p-t-Bucinal), and Cyclopentanone, 2-[2-(4-methyl-3-cyclohexen-1-yl)propyl]- and 1-methyl-4-(1-methylethenyl)cyclohexene and mixtures thereof.

In one aspect the composition may comprise an encapsulated perfume particle comprising either a water-soluble hydroxylic compound or melamine-formaldehyde or modified polyvinyl alcohol. In one aspect the encapsulate comprises (a) an at least partially water-soluble solid matrix comprising one or more water-soluble hydroxylic compounds, preferably starch; and (b) a perfume oil encapsulated by the solid matrix.

In a further aspect the perfume may be pre-complexed with a polyamine, preferably a polyethylenimine so as to form a Schiff base.

Polymers—

The composition may comprise one or more polymers. Examples are carboxymethylcellulose, poly(vinyl-pyrrolidone), poly (ethylene glycol), poly(vinyl alcohol), poly(vinylpyridine-N-oxide), poly(vinylimidazole), polycarboxylates such as polyacrylates, maleic/acrylic acid copolymers and lauryl methacrylate/acrylic acid co-polymers.

The composition may comprise one or more amphiphilic cleaning polymers such as the compound having the following general structure: bis((C₂H₅O)(C₂H₄O)n)(CH₃)—N⁺—CₓH₂ₓ—N⁺—(CH₃)-bis((C₂H₅O)(C₂H₄O)n), wherein n=from 20 to 30, and x=from 3 to 8, or sulphated or sulphonated variants thereof.

The composition may comprise amphiphilic alkoxylated grease cleaning polymers which have balanced hydrophilic and hydrophobic properties such that they remove grease particles from fabrics and surfaces. Specific embodiments of the amphiphilic alkoxylated grease cleaning polymers of the present invention comprise a core structure and a plurality of alkoxylate groups attached to that core structure. These may comprise alkoxylated polyalkylenimines, preferably having an inner polyethylene oxide block and an outer polypropylene oxide block.

Alkoxylated polycarboxylates such as those prepared from polyacrylates are useful herein to provide additional grease removal performance. Such materials are described in WO91/08281 and PCT90/01815. Chemically, these materials comprise polyacrylates having one ethoxy side-chain per every 7-8 acrylate units. The side-chains are of the formula —(CH₂CH₂O)ₘ, (CH₂)ₙCH₃ wherein m is 2-3 and n is 6-12. The side-chains are ester-linked to the polyacrylate “backbone” to provide a “comb” polymer type structure. The molecular weight can vary, but is typically in the range of 2000 to 50,000. Such alkoxylated polycarboxylates can comprise from 0.05 wt % to 10 wt % of the compositions herein.

The isoprenoid-derived surfactants of the present invention, and their mixtures with other cosurfactants and other adjunct ingredients, are particularly suited to be used with an amphilic graft co-polymer, preferably the amphilic graft co-polymer comprises (i) polyethyelene glycol backbone; and (ii) and at least one pendant moiety selected from polyvinyl acetate, polyvinyl alcohol and mixtures thereof. A preferred amphilic graft co-polymer is Sokalan HP22, supplied from BASF. Suitable polymers include random graft copolymers, preferably a polyvinyl acetate grafted polyethylene oxide copolymer having a polyethylene oxide backbone and multiple polyvinyl acetate side chains. The molecular weight of the polyethylene oxide backbone is preferably 6000 and the weight ratio of the polyethylene oxide to polyvinyl acetate is 40 to 60 and no more than 1 grafting point per 50 ethylene oxide units.

Carboxylate Polymer—

The composition of the present invention may also include one or more carboxylate polymers such as a maleate/acrylate random copolymer or polyacrylate homopolymer. In one aspect, the carboxylate polymer is a polyacrylate homopolymer having a molecular weight of from 4,000 to 9,000 Da, or from 6,000 to 9,000 Da.

Soil Release Polymer—

The composition of the present invention may also include one or more soil release polymers having a structure as defined by one of the following structures (I), (II) or (III):

—[(OCHR¹—CHR²)ₐ—O—O—C—Ar—CO—]_{d} (I)

—[(OCHR³—CHR⁴)_{b}—O—OC-sAr—CO—]ₑ (II)

—[(OCHR⁵—CHR⁶)_{c}—OR⁷]_{f} (III)

wherein:
a, b and c are from 1 to 200;
d, e and f are from 1 to 50;
Ar is a 1,4-substituted phenylene;
sAr is 1,3-substituted phenylene substituted in position 5 with SO₃Me;
Me is Li, K, Mg/2, Ca/2, Al/3, ammonium, mono-, di-, tri-, or tetraalkylammonium wherein the alkyl groups are C₁-C₁₈ alkyl or C₂-C₁₀ hydroxyalkyl, or mixtures thereof;
R¹, R², R³, R⁴, R⁵ and R⁶ are independently selected from H or C₁-C₁₈ n- or iso-alkyl; and
R⁷ is a linear or branched C₁-C₁₈ alkyl, or a linear or branched C₂-C₃₀ alkenyl, or a cycloalkyl group with 5 to 9 carbon atoms, or a C₈-C₃₀ aryl group, or a C₆-C₃₀ arylalkyl group.

Suitable soil release polymers are polyester soil release polymers such as Repel-o-tex polymers, including Repel-o-tex, SF-2 and SRP6 supplied by Rhodia. Other suitable soil release polymers include Texcare polymers, including Texcare SRA100, SRA300, SRN100, SRN170, SRN240, SRN300 and SRN325 supplied by Clariant. Other suitable soil release polymers are Marloquest polymers, such as Marloquest SL supplied by Sasol.

Cellulosic Polymer—

The composition of the present invention may also include one or more cellulosic polymers including those selected from alkyl cellulose, alkyl alkoxyalkyl cellulose, carboxyalkyl cellulose, alkyl carboxyalkyl cellulose. In one aspect, the cellulosic polymers are selected from the group comprising carboxymethyl cellulose, methyl cellulose, methyl hydroxyethyl cellulose, methyl carboxymethyl cellulose, and mixtures thereof. In one aspect, the carboxymethyl cellulose has a degree of carboxymethyl substitution from 0.5 to 0.9 and a molecular weight from 100,000 to 300,000 Da.

Enzymes—

The composition may comprise one or more additional enzymes which provide cleaning performance and/or fabric care benefits. Examples of suitable enzymes include, but are not limited to, hemicellulases, peroxidases, proteases, cellulases, xylanases, lipases, phospholipases, esterases, cutinases, pectinases, mannanases, pectate lyases, keratinases, reductases, oxidases, phenoloxidases, lipoxygenases, xanthanase, ligninases, pullulanases, tannases, pentosanases, malanases, β-glucanases, arabinosidases, hyaluronidase, chondroitinase, laccase, chlorophyllases and amylases, or mixtures thereof. A typical combination is an enzyme cocktail that may comprise e.g. a protease and lipase in conjunction with amylase. When present in a composition, the aforementioned additional enzymes may be present at levels from 0.00001 to 2 wt %, from 0.0001 to 1 wt % or from 0.001 to 0.5 wt % enzyme protein by weight of the composition.

In general the properties of the selected enzyme(s) should be compatible with the selected detergent, (i.e., pH-optimum, compatibility with other enzymatic and non-enzymatic ingredients, etc.), and the enzyme(s) should be present in effective amounts.

Cellulases—

Suitable cellulases include those of bacterial or fungal origin. Chemically modified or protein engineered mutants are included. Suitable cellulases include cellulases from the genera Bacillus, Pseudomonas, Humicola, Fusarium, Thielavia, Acremonium, e.g., the fungal cellulases produced from Humicola insolens, Myceliophthora thermophila and Fusarium oxysporum disclosed in U.S. Pat. No. 4,435,307, U.S. Pat. No. 5,648,263, U.S. Pat. No. 5,691,178, U.S. Pat. No. 5,776,757 and WO89/09259.

Especially suitable cellulases are the alkaline or neutral cellulases having colour care benefits. Examples of such cellulases are cellulases described in EPO495257, EP0531372, WO96/11262, WO96/29397, and WO98/08940. Other examples are cellulase variants such as those described in WO94/07998, EP0531315, U.S. Pat. No. 5,457,046, U.S. Pat. No. 5,686,593, U.S. Pat. No. 5,763,254, WO95/24471, WO98/12307 and WO99/001544.

Other cellulases are endo-beta-1,4-glucanase enzyme having a sequence of at least 97% identity to the amino acid sequence of position 1 to position 773 of SEQ ID NO:2 of WO02/099091 or a family 44 xyloglucanase, which a xyloglucanase enzyme having a sequence of at least 60% identity to positions 40-559 of SEQ ID NO: 2 of WO01/062903.

Commercially available cellulases include Celluzyme™, and Carezyme™ (Novozymes A/S) Carezyme Premium™ (Novozymes A/S), Celluclean™ (Novozymes A/S), Celluclean Classic™ (Novozymes A/S), Cellusoft™ (Novozymes A/S), Whitezyme™ (Novozymes A/S), Clazinase™, and Puradax HA™ (Genencor International Inc.), and KAC-500(B)™ (Kao Corporation).

Proteases—

Suitable proteases include those of bacterial, fungal, plant, viral or animal origin e.g. vegetable or microbial origin. Microbial origin is preferred. Chemically modified or protein engineered mutants are included. It may be an alkaline protease, such as a serine protease or a metalloprotease. A serine protease may for example be of the 51 family, such as trypsin, or the S8 family such as subtilisin. A metalloproteases protease may for example be a thermolysin from e.g. family M4 or other metalloprotease such as those from M5, M7 or M8 families.

The term “subtilases” refers to a sub-group of serine protease according to Siezen et al., Protein Engng. 4 (1991) 719-737 and Siezen et al. Protein Science 6 (1997) 501-523. Serine proteases are a subgroup of proteases characterized by having a serine in the active site, which forms a covalent adduct with the substrate. The subtilases may be divided into 6 sub-divisions, i.e. the Subtilisin family, the Thermitase family, the Proteinase K family, the Lantibiotic peptidase family, the Kexin family and the Pyrolysin family.

Examples of subtilases are those derived from Bacillus such as Bacillus lentus, B. alkalophilus, B. subtilis, B. amyloliquefaciens, Bacillus pumilus and Bacillus gibsonii described in; U.S. Pat. No. 7,262,042 and WO09/021867, and subtilisin lentus, subtilisin Novo, subtilisin Carlsberg, Bacillus licheniformis, subtilisin BPN′, subtilisin 309, subtilisin 147 and subtilisin 168 described in WO89/06279 and protease PD138 described in (WO93/18140). Other useful proteases may be those described in WO92/175177, WO01/016285, WO02/026024 and WO02/016547. Examples of trypsin-like proteases are trypsin (e.g. of porcine or bovine origin) and the Fusarium protease described in WO89/06270, WO94/25583 and WO05/040372, and the chymotrypsin proteases derived from Cellumonas described in WO05/052161 and WO05/052146.

A further preferred protease is the alkaline protease from Bacillus lentus DSM 5483, as described for example in WO95/23221, and variants thereof which are described in WO92/21760, WO95/23221, EP1921147 and EP1921148.

Examples of metalloproteases are the neutral metalloprotease as described in WO07/044993 (Genencor Int.) such as those derived from Bacillus amyloliquefaciens.

Examples of useful proteases are the variants described in: WO92/19729, WO96/034946, WO98/20115, WO98/20116, WO99/011768, WO01/44452, WO03/006602, WO04/03186, WO04/041979, WO07/006305, WO11/036263, WO11/036264, especially the variants with substitutions in one or more of the following positions: 3, 4, 9, 15, 27, 36, 57, 68, 76, 87, 95, 96, 97, 98, 99, 100, 101, 102, 103, 104, 106, 118, 120, 123, 128, 129, 130, 160, 167, 170, 194, 195, 199, 205, 206, 217, 218, 222, 224, 232, 235, 236, 245, 248, 252 and 274 using the BPN′ numbering. More preferred the subtilase variants may comprise the mutations: S3T, V41, S9R, A15T, K27R, *36D, V68A, N76D, N87S,R, *97E, A98S, S99G,D,A, S99AD, S101G,M,R S103A, V1041,Y,N, S106A, G118V,R, H120D,N, N123S, 5128L, P129Q, 5130A, G160D, Y167A, R1705, A194P, G195E, V199M, V2051, L217D, N218D, M222S, A232V, K235L, Q236H, Q245R, N252K, T274A (using BPN′ numbering).

Suitable commercially available protease enzymes include those sold under the trade names Alcalase®, Duralase™, Durazym™, Relase®, Relase® Ultra, Savinase®, Savinase® Ultra, Primase®, Polarzyme®, Kannase®, Liquanase®, Liquanase® Ultra, Ovozyme®, Coronase®, Coronase® Ultra, Neutrase®, Everlase® and Esperase® (Novozymes A/S), those sold under the tradename Maxatase®, Maxacal®, Maxapem®, Purafect®, Purafect Prime®, Preferenz™, Purafect MA®, Purafect Ox®, Purafect OxP®, Puramax®, Properase®, Effectenz™, FN2®, FN3®, FN4®, Excellase®, Opticlean® and Optimase® (Danisco/DuPont), Axapem™ (Gist-Brocases N.V.), BLAP (sequence shown in FIG. 29 of U.S. Pat. No. 5,352,604) and variants hereof (Henkel AG) and KAP (Bacillus alkalophilus subtilisin) from Kao.

Lipases and Cutinases—

Suitable lipases and cutinases include those of bacterial or fungal origin. Chemically modified or protein engineered mutant enzymes are included. Examples include lipase from Thermomyces, e.g. from T. lanuginosus (previously named Humicola lanuginosa) as described in EP258068 and EP305216, cutinase from Humicola, e.g. H. insolens (WO96/13580), lipase from strains of Pseudomonas (some of these now renamed to Burkholderia), e.g. P. alcaligenes or P. pseudoalcaligenes (EP218272), P. cepacia (EP331376), P. sp. strain SD705 (WO95/06720 & WO96/27002), P. wisconsinensis (WO96/12012), GDSL-type Streptomyces lipases (WO10/065455), cutinase from Magnaporthe grisea (WO10/107560), cutinase from Pseudomonas mendocina (U.S. Pat. No. 5,389,536), lipase from Thermobifida fusca (WO11/084412), Geobacillus stearothermophilus lipase (WO11/084417), lipase from Bacillus subtilis (WO11/084599), and lipase from Streptomyces griseus (WO11/150157) and S. pristinaespiralis (WO12/137147).

Other examples are lipase variants such as those described in EP407225, WO92/05249, WO94/01541, WO94/25578, WO95/14783, WO95/30744, WO95/35381, WO95/22615, WO96/00292, WO97/04079, WO97/07202, WO00/34450, WO00/60063, WO01/92502, WO07/87508 and WO09/109500.

Preferred commercial lipase products include Lipolase™, Lipex™; Lipolex™ and Lipoclean™ (Novozymes A/S), Lumafast (originally from Genencor) and Lipomax (originally from Gist-Brocades).

Still other examples are lipases sometimes referred to as acyltransferases or perhydrolases, e.g. acyltransferases with homology to Candida antarctica lipase A (WO10/111143), acyltransferase from Mycobacterium smegmatis (WO05/56782), perhydrolases from the CE 7 family (WO09/67279), and variants of the M. smegmatis perhydrolase in particular the S54V variant used in the commercial product Gentle Power Bleach from Huntsman Textile Effects Pte Ltd (WO10/100028).

Amylases—

Suitable amylases which can be used together with the enzyme/variant/blend of enzymes of the invention may be an alpha-amylase or a glucoamylase and may be of bacterial or fungal origin. Chemically modified or protein engineered mutants are included. Amylases include, for example, alpha-amylases obtained from Bacillus, e.g., a special strain of Bacillus licheniformis, described in more detail in GB1296839.

Suitable amylases include amylases having SEQ ID NO: 2 in WO95/10603 or variants having 90% sequence identity to SEQ ID NO: 3 thereof. Preferred variants are described in WO94/02597, WO94/18314, WO97/43424 and SEQ ID NO: 4 of WO99/019467, such as variants with substitutions in one or more of the following positions: 15, 23, 105, 106, 124, 128, 133, 154, 156, 178, 179, 181, 188, 190, 197, 201, 202, 207, 208, 209, 211, 243, 264, 304, 305, 391, 408, and 444.

Different suitable amylases include amylases having SEQ ID NO: 6 in WO02/010355 or variants thereof having 90% sequence identity to SEQ ID NO: 6. Preferred variants of SEQ ID NO: 6 are those having a deletion in positions 181 and 182 and a substitution in position 193.

Other amylases which are suitable are hybrid alpha-amylase comprising residues 1-33 of the alpha-amylase derived from B. amyloliquefaciens shown in SEQ ID NO: 6 of WO06/066594 and residues 36-483 of the B. licheniformis alpha-amylase shown in SEQ ID NO: 4 of WO06/066594 or variants having 90% sequence identity thereof. Preferred variants of this hybrid alpha-amylase are those having a substitution, a deletion or an insertion in one of more of the following positions: G48, T49, G107, H156, A181, N190, M197, 1201, A209 and Q264. Most preferred variants of the hybrid alpha-amylase comprising residues 1-33 of the alpha-amylase derived from B. amyloliquefaciens shown in SEQ ID NO: 6 of WO06/066594 and residues 36-483 of SEQ ID NO: 4 are those having the substitutions:

### M197T;

### H156Y+A181T+N190F+A209V+Q264S; or

### G48A+T491I+G107A+H156Y+A181T+N190F+1201F+A209V+Q264S.

Further amylases which are suitable are amylases having SEQ ID NO: 6 in WO99/019467 or variants thereof having 90% sequence identity to SEQ ID NO: 6. Preferred variants of SEQ ID NO: 6 are those having a substitution, a deletion or an insertion in one or more of the following positions: R181, G182, H183, G184, N195, 1206, E212, E216 and K269. Particularly preferred amylases are those having deletion in positions R181 and G182, or positions H183 and G184.

Additional amylases which can be used are those having SEQ ID NO: 1, SEQ ID NO: 3, SEQ ID NO: 2 or SEQ ID NO: 7 of WO96/023873 or variants thereof having 90% sequence identity to SEQ ID NO: 1, SEQ ID NO: 2, SEQ ID NO: 3 or SEQ ID NO: 7. Preferred variants of SEQ ID NO: 1, SEQ ID NO: 2, SEQ ID NO: 3 or SEQ ID NO: 7 are those having a substitution, a deletion or an insertion in one or more of the following positions: 140, 181, 182, 183, 184, 195, 206, 212, 243, 260, 269, 304 and 476, using SEQ ID 2 of WO96/023873 for numbering. More preferred variants are those having a deletion in two positions selected from 181, 182, 183 and 184, such as 181 and 182, 182 and 183, or positions 183 and 184. Most preferred amylase variants of SEQ ID NO: 1, SEQ ID NO: 2 or SEQ ID NO: 7 are those having a deletion in positions 183 and 184 and a substitution in one or more of positions 140, 195, 206, 243, 260, 304 and 476.

Other amylases which can be used are amylases having SEQ ID NO: 2 of WO08/153815, SEQ ID NO: 10 in WO 01/66712 or variants thereof having 90% sequence identity to SEQ ID NO: 2 of WO08/153815 or 90% sequence identity to SEQ ID NO: 10 in WO01/66712. Preferred variants of SEQ ID NO: 10 in WO01/66712 are those having a substitution, a deletion or an insertion in one of more of the following positions: 176, 177, 178, 179, 190, 201, 207, 211 and 264.

Further suitable amylases are amylases having SEQ ID NO: 2 of WO09/061380 or variants having 90% sequence identity to SEQ ID NO: 2 thereof. Preferred variants of SEQ ID NO: 2 are those having a truncation of the C-terminus and/or a substitution, a deletion or an insertion in one of more of the following positions: Q87, Q98, S125, N128, T131, T165, K178, R180, S181, T182, G183, M201, F202, N225, S243, N272, N282, Y305, R309, D319, Q320, Q359, K444 and G475. More preferred variants of SEQ ID NO: 2 are those having the substitution in one of more of the following positions: Q87E,R, Q98R, S125A, N128C, T131I, T165I, K178L, T182G, M201L, F202Y, N225E,R, N272E,R, S243Q,A,E,D, Y305R, R309A, Q320R, Q359E, K444E and G475K and/or deletion in position R180 and/or S181 or of T182 and/or G183. Most preferred amylase variants of SEQ ID NO: 2 are those having the substitutions:

### N128C+K178L+T182G+Y305R+G475K;

### N128C+K178L+T182G+F202Y+Y305R+D319T+G475K;

### S125A+N128C+K178L+T182G+Y305R+G475K; or

S125A+N128C+T131I+T165I+K178L+T182G+Y305R+G475K wherein the variants are C-terminally truncated and optionally further comprises a substitution at position 243 and/or a deletion at position 180 and/or position 181.

Other suitable amylases are the alpha-amylase having SEQ ID NO: 12 in WO01/66712 or a variant having at least 90% sequence identity to SEQ ID NO: 12. Preferred amylase variants are those having a substitution, a deletion or an insertion in one of more of the following positions of SEQ ID NO: 12 in WO01/66712: R28, R118, N174; R181, G182, D183, G184, G186, W189, N195, M202, Y298, N299, K302, S303, N306, R310, N314; R320, H324, E345, Y396, R400, W439, R444, N445, K446, Q449, R458, N471, N484. Particular preferred amylases include variants having a deletion of D183 and G184 and having the substitutions R118K, N195F, R320K and R458K, and a variant additionally having substitutions in one or more position selected from the group: M9, G149, G182, G186, M202, T257, Y295, N299, M323, E345 and A339, most preferred a variant that additionally has substitutions in all these positions.

Other examples are amylase variants such as those described in WO2011/098531, WO2013/001078 and WO2013/001087.

Commercially available amylases are Duramyl™, Termamyl™, Fungamyl™, Stainzyme™, Stainzyme Plus™, Natalase™, Liquozyme X and BAN™ (from Novozymes A/S), and Rapidase™, Purastar™/Effectenz™, Powerase and Preferenz S100 (from Genencor International Inc./Du Pont).

Peroxidases/Oxidases—

Suitable peroxidases according to the invention is a peroxidase enzyme comprised by the enzyme classification EC 1.11.1.7, as set out by the Nomenclature Committee of the International Union of Biochemistry and Molecular Biology (IUBMB), or any fragment derived therefrom, exhibiting peroxidase activity.

Suitable peroxidases include those of plant, bacterial or fungal origin. Chemically modified or protein engineered mutants are included. Examples of useful peroxidases include peroxidases from Coprinopsis, e.g., from C. cinerea (EP179486), and variants thereof as those described in WO93/24618, WO95/10602, and WO98/15257.

A peroxidase also includes a haloperoxidase enzyme, such as chloroperoxidase, bromoperoxidase and compounds exhibiting chloroperoxidase or bromoperoxidase activity. Haloperoxidases are classified according to their specificity for halide ions. Chloroperoxidases (E.C. 1.11.1.10) catalyze formation of hypochlorite from chloride ions.

In an embodiment, the haloperoxidase of the invention is a chloroperoxidase. Preferably, the haloperoxidase is a vanadium haloperoxidase, i.e., a vanadate-containing haloperoxidase. In a preferred method of the present invention the vanadate-containing haloperoxidase is combined with a source of chloride ion.

Haloperoxidases have been isolated from many different fungi, in particular from the fungus group dematiaceous hyphomycetes, such as Caldariomyces, e.g., C. fumago, Alternaria, Curvularia, e.g., C. verruculosa and C. inaequalis, Drechslera, Ulocladium and Botrytis.

Haloperoxidases have also been isolated from bacteria such as Pseudomonas, e.g., P. pyrrocinia and Streptomyces, e.g., S. aureofaciens.

In an preferred embodiment, the haloperoxidase is derivable from Curvularia sp., in particular Curvularia verruculosa or Curvularia inaequalis, such as C. inaequalis CBS 102.42 as described in WO95/27046; or C. verruculosa CBS 147.63 or C. verruculosa CBS 444.70 as described in WO97/04102; or from Drechslera hartlebii as described in WO01/79459, Dendryphiella salina as described in WO01/79458, Phaeotrichoconis crotalarie as described in WO01/79461, or Geniculosporium sp. as described in WO01/79460.

An oxidase according to the invention include, in particular, any laccase enzyme comprised by the enzyme classification EC 1.10.3.2, or any fragment derived therefrom exhibiting laccase activity, or a compound exhibiting a similar activity, such as a catechol oxidase (EC 1.10.3.1), an o-aminophenol oxidase (EC 1.10.3.4), or a bilirubin oxidase (EC 1.3.3.5).

Preferred laccase enzymes are enzymes of microbial origin. The enzymes may be derived from plants, bacteria or fungi (including filamentous fungi and yeasts).

Suitable examples from fungi include a laccase derivable from a strain of Aspergillus, Neurospora, e.g., N. crassa, Podospora, Botrytis, Collybia, Fomes, Lentinus, Pleurotus, Trametes, e.g., T. villosa and T. versicolor, Rhizoctonia, e.g., R. solani, Coprinopsis, e.g., C. cinerea, C. comatus, C. friesii, and C. plicatilis, Psathyrella, e.g., P. condelleana, Panaeolus, e.g., P. papilionaceus, Myceliophthora, e.g., M. thermophila, Schytalidium, e.g., S. thermophilum, Polyporus, e.g., P. pinsitus, Phlebia, e.g., P. radiata (WO92/01046), or Coriolus, e.g., C. hirsutus (JP2238885).

Suitable examples from bacteria include a laccase derivable from a strain of Bacillus. A laccase derived from Coprinopsis or Myceliophthora is preferred; in particular a laccase derived from Coprinopsis cinerea, as disclosed in WO97/08325; or from Myceliophthora thermophila, as disclosed in WO95/33836.

Other preferred enzymes include pectate lyases sold under the tradenames Pectawash®, Pectaway®, Xpect® and mannanases sold under the tradenames Mannaway® (Novozymes), and Purabrite® (Danisco/Dupont).

The detergent enzyme(s) may be included in a detergent composition by adding separate additives containing one or more enzymes, or by adding a combined additive comprising all of these enzymes. A detergent additive of the invention, i.e., a separate additive or a combined additive, can be formulated, for example, as a granulate, liquid, slurry, etc. Preferred detergent additive formulations are granulates, in particular non-dusting granulates, liquids, in particular stabilized liquids, or slurries.

Non-dusting granulates may be produced, e.g. as disclosed in U.S. Pat. No. 4,106,991 and U.S. Pat. No. 4,661,452 and may optionally be coated by methods known in the art. Examples of waxy coating materials are poly(ethylene oxide) products (polyethyleneglycol, PEG) with mean molar weights of 1000 to 20000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in GB1483591. Liquid enzyme preparations may, for instance, be stabilized by adding a polyol such as propylene glycol, a sugar or sugar alcohol, lactic acid or boric acid according to established methods. Protected enzymes may be prepared according to the method disclosed in EP238216.

Dye Transfer Inhibiting Agents—

The compositions of the present invention may also include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. When present in a composition, the dye transfer inhibiting agents may be present at levels from 0.0001 to 10 wt %, from 0.01 to 5 wt % or from 0.1 to 3 wt %.

Brighteners—

The compositions of the present invention can also contain additional components that may tint articles being cleaned, such as fluorescent brighteners. The composition may comprise C.I. fluorescent brightener 260 in alpha-crystalline form having the following structure:

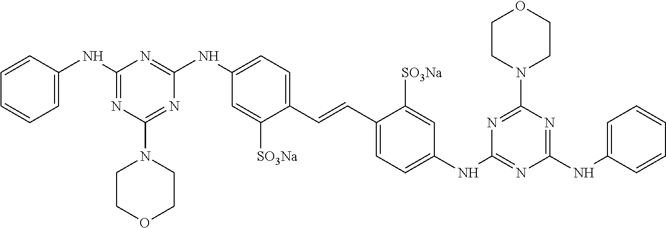

In one aspect, the brightener is a cold water soluble brightener, such as the C.I. fluorescent brightener 260 in alpha-crystalline form. In one aspect the brightener is predominantly in alpha-crystalline form, which means that typically at least 50 wt %, at least 75 wt %, at least 90 wt %, at least 99 wt %, or even substantially all, of the C.I. fluorescent brightener 260 is in alpha-crystalline form.

The brightener is typically in micronized particulate form, having a weight average primary particle size of from 3 to 30 micrometers, from 3 micrometers to 20 micrometers, or from 3 to 10 micrometers.

The composition may comprise C.I. fluorescent brightener 260 in beta-crystalline form, and the weight ratio of: (i) C.I. fluorescent brightener 260 in alpha-crystalline form, to (ii) C.I. fluorescent brightener 260 in beta-crystalline form may be at least 0.1, or at least 0.6. BE680847 relates to a process for making 0.1 fluorescent brightener 260 in alpha-crystalline form.

Commercial optical brighteners which may be useful in the present invention can be classified into subgroups, which include, but are not necessarily limited to, derivatives of stilbene, pyrazoline, coumarin, carboxylic acid, methinecyanines, dibenzothiophene-5,5-dioxide, azoles, 5- and 6-membered-ring heterocycles, and other miscellaneous agents. Examples of such brighteners are disclosed in “The Production and Application of Fluorescent Brightening Agents”, M. Zahradnik, Published by John Wiley & Sons, New York (1982). Specific nonlimiting examples of optical brighteners which are useful in the present compositions are those identified in U.S. Pat. No. 4,790,856 and U.S. Pat. No. 3,646,015.

A further suitable brightener has the structure below:

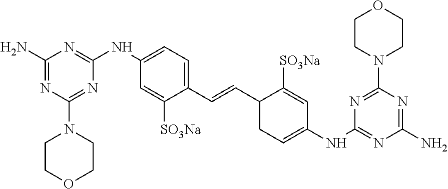

Suitable fluorescent brightener levels include lower levels of from 0.01 wt %, from 0.05 wt %, from 0.1 wt % or from 0.2 wt % to upper levels of 0.5 wt % or 0.75 wt %. In one aspect the brightener may be loaded onto a clay to form a particle. Silicate salts—The compositions of the present invention can also contain silicate salts, such as sodium or potassium silicate. The composition may comprise of from 0 wt % to less than 10 wt % silicate salt, to 9 wt %, or to 8 wt %, or to 7 wt %, or to 6 wt %, or to 5 wt %, or to 4 wt %, or to 3 wt %, or even to 2 wt %, and from above 0 wt %, or from 0.5 wt %, or from 1 wt % silicate salt. A suitable silicate salt is sodium silicate.

Dispersants—

The compositions of the present invention can also contain dispersants. Suitable water-soluble organic materials include the homo- or co-polymeric acids or their salts, in which the polycarboxylic acid comprises at least two carboxyl radicals separated from each other by not more than two carbon atoms.

Enzyme Stabilizers—

Enzymes for use in compositions can be stabilized by various techniques. The enzymes employed herein can be stabilized by the presence of water-soluble sources of calcium and/or magnesium ions. Examples of conventional stabilizing agents are, e.g. a polyol such as propylene glycol or glycerol, a sugar or sugar alcohol, lactic acid, boric acid, or a boric acid derivative, e.g. an aromatic borate ester, or a phenyl boronic acid derivative such as 4-formylphenyl boronic acid, and the composition may be formulated as described in, for example, WO92/19709 and WO92/19708 In case of aqueous compositions comprising protease, a reversible protease inhibitor, such as a boron compound including borate, 4-formyl phenylboronic acid, phenylboronic acid and derivatives thereof, or compounds such as calcium formate, sodium formate and 1,2-propane diol can be added to further improve stability.

Solvents—

Suitable solvents include water and other solvents such as lipophilic fluids. Examples of suitable lipophilic fluids include siloxanes, other silicones, hydrocarbons, glycol ethers, glycerine derivatives such as glycerine ethers, perfluorinated amines, perfluorinated and hydrofluoroether solvents, low-volatility nonfluorinated organic solvents, diol solvents, other environmentally-friendly solvents and mixtures thereof.

Structurant/Thickeners—

Structured liquids can either be internally structured, whereby the structure is formed by primary ingredients (e.g. surfactant material) and/or externally structured by providing a three dimensional matrix structure using secondary ingredients (e.g. polymers, clay and/or silicate material). The composition may comprise a structurant, from 0.01 to 5 wt %, or from 0.1 to 2.0 wt %. The structurant is typically selected from the group consisting of diglycerides and triglycerides, ethylene glycol distearate, microcrystalline cellulose, cellulose-based materials, microfiber cellulose, hydrophobically modified alkali-swellable emulsions such as Polygel W30 (3VSigma), biopolymers, xanthan gum, gellan gum, and mixtures thereof. A suitable structurant includes hydrogenated castor oil, and non-ethoxylated derivatives thereof. A suitable structurant is disclosed in U.S. Pat. No. 6,855,680. Such structurants have a thread-like structuring system having a range of aspect ratios. Other suitable structurants and the processes for making them are described in WO10/034736.

Conditioning Agents—

The composition of the present invention may include a high melting point fatty compound. The high melting point fatty compound useful herein has a melting point of 25° C. or higher, and is selected from the group consisting of fatty alcohols, fatty acids, fatty alcohol derivatives, fatty acid derivatives, and mixtures thereof. Such compounds of low melting point are not intended to be included in this section. Non-limiting examples of the high melting point compounds are found in International Cosmetic Ingredient Dictionary, Fifth Edition, 1993, and CTFA Cosmetic Ingredient Handbook, Second Edition, 1992.

The high melting point fatty compound is included in the composition at a level of from 0.1 to 40 wt %, from 1 to 30 wt %, from 1.5 to 16 wt %, from 1.5 to 8 wt % in view of providing improved conditioning benefits such as slippery feel during the application to wet hair, softness and moisturized feel on dry hair.

The compositions of the present invention may contain a cationic polymer. Concentrations of the cationic polymer in the composition typically range from 0.05 to 3 wt %, from 0.075 to 2.0 wt %, or from 0.1 to 1.0 wt %. Suitable cationic polymers will have cationic charge densities of at least 0.5 meq/gm, at least 0.9 meq/gm, at least 1.2 meq/gm, at least 1.5 meq/gm, or less than 7 meq/gm, and less than 5 meq/gm, at the pH of intended use of the composition, which pH will generally range from pH3 to pH9, or between pH4 and pH8. Herein, “cationic charge density” of a polymer refers to the ratio of the number of positive charges on the polymer to the molecular weight of the polymer. The average molecular weight of such suitable cationic polymers will generally be between 10,000 and 10 million, between 50,000 and 5 million, or between 100,000 and 3 million.

Suitable cationic polymers for use in the compositions of the present invention contain cationic nitrogen-containing moieties such as quaternary ammonium or cationic protonated amino moieties. Any anionic counterions can be used in association with the cationic polymers so long as the polymers remain soluble in water, in the composition, or in a coacervate phase of the composition, and so long as the counterions are physically and chemically compatible with the essential components of the composition or do not otherwise unduly impair composition performance, stability or aesthetics. Nonlimiting examples of such counterions include halides (e.g., chloride, fluoride, bromide, iodide), sulfate and methylsulfate.

Nonlimiting examples of such polymers are described in the CTFA Cosmetic Ingredient Dictionary, 3rd edition, edited by Estrin, Crosley, and Haynes, (The Cosmetic, Toiletry, and Fragrance Association, Inc., Washington, D.C. (1982)).

Other suitable cationic polymers for use in the composition include polysaccharide polymers, cationic guar gum derivatives, quaternary nitrogen-containing cellulose ethers, synthetic polymers, copolymers of etherified cellulose, guar and starch. When used, the cationic polymers herein are either soluble in the composition or are soluble in a complex coacervate phase in the composition formed by the cationic polymer and the anionic, amphoteric and/or zwitterionic surfactant component described hereinbefore. Complex coacervates of the cationic polymer can also be formed with other charged materials in the composition. Suitable cationic polymers are described in U.S. Pat. No. 3,962,418; U.S. Pat. No. 3,958,581; and US2007/0207109.

The composition of the present invention may include a nonionic polymer as a conditioning agent. Polyalkylene glycols having a molecular weight of more than 1000 are useful herein Useful are those having the following general formula:

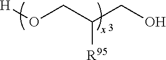

wherein R⁹⁵ is selected from the group consisting of H, methyl, and mixtures thereof. Conditioning agents, and in particular silicones, may be included in the composition. The conditioning agents useful in the compositions of the present invention typically comprise a water insoluble, water dispersible, non-volatile, liquid that forms emulsified, liquid particles. Suitable conditioning agents for use in the composition are those conditioning agents characterized generally as silicones (e.g., silicone oils, cationic silicones, silicone gums, high refractive silicones, and silicone resins), organic conditioning oils (e.g., hydrocarbon oils, polyolefins, and fatty esters) or combinations thereof, or those conditioning agents which otherwise form liquid, dispersed particles in the aqueous surfactant matrix herein. Such conditioning agents should be physically and chemically compatible with the essential components of the composition, and should not otherwise unduly impair composition stability, aesthetics or performance.

The concentration of the conditioning agent in the composition should be sufficient to provide the desired conditioning benefits. Such concentration can vary with the conditioning agent, the conditioning performance desired, the average size of the conditioning agent particles, the type and concentration of other components, and other like factors.

The concentration of the silicone conditioning agent typically ranges from 0.01 to 10 wt %. Non-limiting examples of suitable silicone conditioning agents, and optional suspending agents for the silicone, are described in U.S. Reissue Pat. No. 34,584; U.S. Pat. No. 5,104,646; U.S. Pat. No. 5,106,609; U.S. Pat. No. 4,152,416; U.S. Pat. No. 2,826,551; U.S. Pat. No. 3,964,500; U.S. Pat. No. 4,364,837; U.S. Pat. No. 6,607,717; U.S. Pat. No. 6,482,969; U.S. Pat. No. 5,807,956; U.S. Pat. No. 5,981,681; U.S. Pat. No. 6,207,782; U.S. Pat. No. 7,465,439; U.S. Pat. No. 7,041,767; U.S. Pat. No. 7,217,777; US2007/0286837A1; US2005/0048549A1; US2007/0041929A1; GB849433; DE10036533, which are all incorporated herein by reference; Chemistry and Technology of Silicones, New York: Academic Press (1968); General Electric Silicone Rubber Product Data Sheets SE 30, SE 33, SE 54 and SE 76; Silicon Compounds, Petrarch Systems, Inc. (1984); and in Encyclopedia of Polymer Science and Engineering, vol. 15, 2d ed., pp 204-308, John Wiley & Sons, Inc. (1989).

The compositions of the present invention may also comprise from 0.05 to 3 wt % of a at least one organic conditioning oil as the conditioning agent, either alone or in combination with other conditioning agents, such as the silicones (described herein). Suitable conditioning oils include hydrocarbon oils, polyolefins, and fatty esters. Also suitable for use in the compositions herein are the conditioning agents described in U.S. Pat. No. 5,674,478 and U.S. Pat. No. 5,750,122 or in U.S. Pat. No. 4,529,586; U.S. Pat. No. 4,507,280; U.S. Pat. No. 4,663,158; U.S. Pat. No. 4,197,865; U.S. Pat. No. 4,217,914; U.S. Pat. No. 4,381,919; and U.S. Pat. No. 4,422,853.

Hygiene and Malodour—

The compositions of the present invention may also comprise one or more of zinc ricinoleate, thymol, quaternary ammonium salts such as Bardac®, polyethylenimines (such as Lupasol® from BASF) and zinc complexes thereof, silver and silver compounds, especially those designed to slowly release Ag⁺ or nano-silver dispersions.

Probiotics—

The compositions may comprise probiotics such as those described in WO09/043709.

Suds Boosters—

If high sudsing is desired, suds boosters such as the C₁₀-C₁₆ alkanolamides or C₁₀-C₁₄ alkyl sulphates can be incorporated into the compositions, typically at 1 to 10 wt % levels. The C₁₀-C₁₄ monoethanol and diethanol amides illustrate a typical class of such suds boosters. Use of such suds boosters with high sudsing adjunct surfactants such as the amine oxides, betaines and sultaines noted above is also advantageous. If desired, water-soluble magnesium and/or calcium salts such as MgCl₂, MgSO₄, CaCl₂, CaSO₄ and the like, can be added at levels of, typically, 0.1 to 2 wt %, to provide additional suds and to enhance grease removal performance.

Suds Suppressors—

Compounds for reducing or suppressing the formation of suds can be incorporated into the compositions of the present invention. Suds suppression can be of particular importance in the so-called “high concentration cleaning process” as described in U.S. Pat. No. 4,489,455 and U.S. Pat. No. 4,489,574, and in front-loading—style washing machines. A wide variety of materials may be used as suds suppressors, and suds suppressors are well known to those skilled in the art. See e.g. Kirk Othmer Encyclopedia of Chemical Technology, Third Edition, Volume 7, p. 430-447 (John Wiley & Sons, Inc., 1979). Examples of suds supressors include monocarboxylic fatty acid and soluble salts therein, high molecular weight hydrocarbons such as paraffin, fatty acid esters (e.g., fatty acid triglycerides), fatty acid esters of monovalent alcohols, aliphatic C₁₈-C₄₀ ketones (e.g., stearone), N-alkylated amino triazines, waxy hydrocarbons preferably having a melting point below about 100° C., silicone suds suppressors, and secondary alcohols. Suds supressors are described in U.S. Pat. No. 2,954,347; U.S. Pat. No. 4,265,779; U.S. Pat. No. 4,265,779; U.S. Pat. No. 3,455,839; U.S. Pat. No. 3,933,672; U.S. Pat. No. 4,652,392; U.S. Pat. No. 4,978,471; U.S. Pat. No. 4,983,316; U.S. Pat. No. 5,288,431; U.S. Pat. No. 4,639,489; U.S. Pat. No. 4,749,740; U.S. Pat. No. 4,798,679; U.S. Pat. No. 4,075,118; EP89307851.9; EP150872; and DOS 2,124,526.

For any detergent compositions to be used in automatic laundry washing machines, suds should not form to the extent that they overflow the washing machine. Suds suppressors, when utilized, are preferably present in a “suds suppressing amount. By “suds suppressing amount” is meant that the formulator of the composition can select an amount of this suds controlling agent that will sufficiently control the suds to result in a low-sudsing laundry detergent for use in automatic laundry washing machines.

The compositions herein will generally comprise from 0 to 10 wt % of suds suppressor. When utilized as suds suppressors, monocarboxylic fatty acids, and salts therein, will be present typically in amounts up to 5 wt %. Preferably, from 0.5 to 3 wt % of fatty monocarboxylate suds suppressor is utilized. Silicone suds suppressors are typically utilized in amounts up to 2.0 wt %, although higher amounts may be used. Monostearyl phosphate suds suppressors are generally utilized in amounts ranging from 0.1 to 2 wt %. Hydrocarbon suds suppressors are typically utilized in amounts ranging from 0.01 to 5.0 wt %, although higher levels can be used. The alcohol suds suppressors are typically used at 0.2 to 3 wt %.

The compositions herein may have a cleaning activity over a broad range of pH. In certain embodiments the compositions have cleaning activity from pH4 to pH11.5. In other embodiments, the compositions are active from pH6 to pH11, from pH7 to pH11, from pH8 to pH11, from pH9 to pH11, or from pH10 to pH11.5.

The compositions herein may have cleaning activity over a wide range of temperatures, e.g., from 10° C. or lower to 90° C. Preferably the temperature will be below 50° C. or 40° C. or even 30° C. In certain embodiments, the optimum temperature range for the compositions is from 10° C. to 20° C., from 15° C. to 25° C., from 15° C. to 30° C., from 20° C. to 30° C., from 25° C. to 35° C., from 30° C. to 40° C., from 35° C. to 45° C., or from 40° C. to 50° C.

### Form of the Detergent Composition

The detergent compositions described herein are advantageously employed for example, in laundry applications for private house hold as well as in industrial and institutional cleaning. The compositions of the invention are in particular solid or liquid cleaning and/or treatment compositions. In one aspect the invention relates to a composition, wherein the form of the composition is selected from the group consisting of a regular, compact or concentrated liquid; a gel; a paste; a soap bar; a regular or a compacted powder; a granulated solid; a homogenous or a multilayer tablet with two or more layers (same or different phases); a pouch having one or more compartments; a single or a multi-compartment unit dose form; or any combination thereof.

The form of the composition may separate the components physically from each other in compartments such as e.g. water dissolvable pouches or in different layers of tablets. Thereby negative storage interaction between components can be avoided. Different dissolution profiles of each of the compartments can also give rise to delayed dissolution of selected components in the wash solution.

Pouches can be configured as single or multicompartments. It can be of any form, shape and material which is suitable for hold the composition, e.g. without allowing the release of the composition to release of the composition from the pouch prior to water contact. The pouch is made from water soluble film which encloses an inner volume. Said inner volume can be divided into compartments of the pouch. Preferred films are polymeric materials preferably polymers which are formed into a film or sheet. Preferred polymers, copolymers or derivates thereof are selected polyacrylates, and water soluble acrylate copolymers, methyl cellulose, carboxy methyl cellulose, sodium dextrin, ethyl cellulose, hydroxyethyl cellulose, hydroxypropyl methyl cellulose, malto dextrin, poly methacrylates, most preferably polyvinyl alcohol copolymers and, hydroxypropyl methyl cellulose (HPMC). Preferably the level of polymer in the film for example PVA is at least about 60%. Preferred average molecular weight will typically be about 20,000 to about 150,000. Films can also be of blended compositions comprising hydrolytically degradable and water soluble polymer blends such as polylactide and polyvinyl alcohol (known under the Trade reference M8630 as sold by MonoSol LLC, Indiana, USA) plus plasticisers like glycerol, ethylene glycerol, propylene glycol, sorbitol and mixtures thereof. The pouches can comprise a solid laundry cleaning composition or part components and/or a liquid cleaning composition or part components separated by the water soluble film. The compartment for liquid components can be different in composition than compartments containing solids (US2009/0011970 A1).

Water-Soluble Film—

The compositions of the present invention may also be encapsulated within a water-soluble film. Preferred film materials are preferably polymeric materials. The film material can e.g. be obtained by casting, blow-moulding, extrusion or blown extrusion of the polymeric material, as known in the art. Preferred polymers, copolymers or derivatives thereof suitable for use as pouch material are selected from polyvinyl alcohols, polyvinyl pyrrolidone, polyalkylene oxides, acrylamide, acrylic acid, cellulose, cellulose ethers, cellulose esters, cellulose amides, polyvinyl acetates, polycarboxylic acids and salts, polyaminoacids or peptides, polyamides, polyacrylamide, copolymers of maleic/acrylic acids, polysaccharides including starch and gelatine, natural gums such as xanthum and carragum. More preferred polymers are selected from polyacrylates and water-soluble acrylate copolymers, methylcellulose, carboxymethylcellulose sodium, dextrin, ethylcellulose, hydroxyethyl cellulose, hydroxypropyl methylcellulose, maltodextrin, polymethacrylates, and most preferably selected from polyvinyl alcohols, polyvinyl alcohol copolymers and hydroxypropyl methyl cellulose (HPMC), and combinations thereof. Preferably, the level of polymer in the pouch material, e.g. a PVA polymer, is at least 60 wt %. The polymer can have any weight average molecular weight, preferably from about 1.000 to 1.000.000, from about 10.000 to 300.000, from about 20.000 to 150.000. Mixtures of polymers can also be used as the pouch material.

Naturally, different film material and/or films of different thickness may be employed in making the compartments of the present invention. A benefit in selecting different films is that the resulting compartments may exhibit different solubility or release characteristics.

Preferred film materials are PVA films known under the MonoSol trade reference M8630, M8900, H8779 and those described in U.S. Pat. No. 6,166,117 and U.S. Pat. No. 6,787,512 and PVA films of corresponding solubility and deformability characteristics.

The film material herein can also comprise one or more additive ingredients. For example, it can be beneficial to add plasticizers, e.g. glycerol, ethylene glycol, diethyleneglycol, propylene glycol, sorbitol and mixtures thereof. Other additives include functional detergent additives to be delivered to the wash water, e.g. organic polymeric dispersants, etc.

### EXAMPLES

### Materials

Chemicals used as buffers and substrates were commercial products of at least reagent grade unless otherwise noted. The amount of ingredients is listed as w/w % of active ingredient unless otherwise indicated.

Lipase—

Lipolase® Lipex® and Lipex® Evity® (from Novozymes A/S, Bagsvrd, Denmark) and one experimental lipase (exp. Lipase, SEQ ID NO: 1) were used in the following experiments. The Amino acid sequence of the exp. Lipase is shown below:

EVSQDLFNQFNLFAQYSAAAYCGKNNRAPAGTNITCTANACPEVEKADAT
FLYSFEDSGVGDVTGFLALDNTNKLIVLSFRGSRSIENWIGNLNFELKEI
NDICSGCRGHAGFTSSWRSVADTLRQKVEDAVREHPDYRVVFTGHSLGGA
LATVAGADLRGNKYDIDVFSYGAPRVGNRAFAEFLTVQTGGTLYRITHTN
DIVPRLPPREFGYSHSSPEYWIKSGTLVPVRRRDIVKIEGIDATGGNNQP
NIPSITAHLWYFGLIGTCL.

Fabric—

Knitted cotton fabric (CN-42, Center For Testmaterials BV, the Netherlands) was pre-washed (desized) and cut into pieces of 6 cm×6 cm.

Lipid Stain

Substrate: Lard (5701043145257, 100% fat, Dragsbaek, Denmark), beef fat (100% fat, Center For Testmaterials BV, the Netherlands), soybean oil (6948195800323, Wilmar, China), peanut oil (072036310538, Harris Teeter, USA).

Coloring agent: Beta-carotene (C9750, synthetic, powder, purity ≧93% (UV), Sigma-Aldrich, USA); Lycopene (Lyc-O-Mato, tomato extract from LycoRed company containing 6% lycopene). Chilli (Chili pepper New Mexico, produced by Santa Maria, Bought in Irma, sample registred November 2014); Red peber (Delikatesse paprika, produced by Santa Maria, Bought in Irma, sample registred November 2014).

Color preserving agent: dl-alpha-tocopherol (T3251, synthetic, purity ≧96% (HPLC), Sigma-Aldrich, USA);

Preparation of Swatches of the Invention—

For preparing the colored lipid stain desired amounts of lipase substrate(s) and optional color preserving agent were weighed into a beaker and melted at 70° C. A coloring agent was added and the mixture was stirred at 70° C. until complete dissolution—approximately 30 minutes.

TABLE B
Colored lipid stain compositions

| Colored lipid stain | Ingredients |
|---|---|
| Lard + beta-carotene | 30 g lard + 0.006 g beta-carotene |
| Lard + beta-carotene + dl-alpha-tocopherol | 30 g lard |
| (0.02% tocopherol was used unless | 0.006 g beta-carotene |
| otherwise indicated) | 0.006 g (0.02%) or 0.012 g (0.04%) dl-alpha- |
| | tocopherol |
| Lard/soybean oil + beta-carotene + dl-alpha- | 30 g mixture of lard and soybean oil in a ratio |
| tocopherol | of 1:1 (w:w) |
| | 0.006 g beta-carotene |
| | 0.006 g dl-alpha-tocopherol |
| Beef fat/peanut oil + beta-carotene + dl-alpha- | 30 g mixture of beef fat and peanut oil in a |
| tocopherol | ratio of 1:1 (w:w) |
| | 0.006 g of beta-carotene |
| | 0.006 g of dl-alpha-tocopherol |
| Lard + lycopene | 30 g lard + 0.015 g lycopene (0.05%) |
| Lard + lycopene + dl-alpha-tocopherol | 30 g lard |
| | 0.015 g lycopene (0.05%) |
| | 0.003 g (0.01%); 0.006 g (0.02%) or 0.012 g |
| | (0.04%) dl-alpha-tocopherol |
| Chilli | 30 g lard |
| | 0.075 g Chili |
| | 0.006 g dl-alpha-tocopherol |
| Paprika | 42.5 g lard |
| | 1.12 g Paprika |

200 uL of a colored lipid stain was applied onto the center of the pre-washed fabric where it was allowed to spread naturally for 15 minutes covered with tin foil at room temperature (for preparation of stains comprising lycopene the spreading step was omitted). The spreading step is optional for preparation of swatches. The swatch was heated to 70° C. for 20 minutes after which it was allowed to cool to room temperature covered with tin foil prior to drying in the dark over night at room temperature. The swatches were stored in the dark at 15° C. until use the same or next day. The swatches were vacuum packed for longer storage.

For preparing swatches with various amounts of stain 100 uL, 200 uL, 300 uL or 400 uL of a lipid stain mixture was applied to the fabric.

Commercial Swatches:

### Lard (KC-S-62, Center For Testmaterials BV, the Netherlands)

Soybean oil with pigment (KC-S-78, Center For Testmaterials BV, the Netherlands)
Beef fat, colored (KC-S-61, Center For Testmaterials BV, the Netherlands)
Beef fat, colored (C-S-61, Center For Testmaterials BV, the Netherlands)
Beef fat, soot (C-S-64, Center For Testmaterials BV, the Netherlands)
Lard, colored (C-S-62, Center For Testmaterials BV, the Netherlands)

### Chlorophyll, Vegetable Oil (C-04, Center For Testmaterials BV, the Netherlands)

### Pigment/Vegetable Fat (W-10PF, wfk Testgewebe GmbH, Germany)

### Pigment/Vegetable Fat (W-20PF, wfk Testgewebe GmbH, Germany)

Detergents—

The following detergents used can be composed according to the description herein. The enzyme(s) to be tested with the swatch can then be added to the detergent/detergent base for testing.

TABLE C
Model Detergent O composition

| | Model O: Asia pacific |
|---|---|
| | model detergent |
| Ingredient (Supplier) | (wt % of active ingredient) |
| LAS (Tianzhi Chemical) | 3.80% |
| AEOS (Tianzhi Chemical) | 8.00% |
| Soap | 1.00% |
| AEO (BASF) | 4.00% |
| Deionized water | 80.10% |
| TEA | 0.40% |
| NaOH | 0.60% |
| Sodium citrate | 2.00% |
| CaCl₂•2H₂O | 0.02% |
| 5-chloro-2-methyl-4-isothiazolin- | 0.10% |
| 3-one & 2-methyl-4-isothiazolin-3- | |
| one (Sunpu Biochemical) | |

TABLE D
Model Detergent H composition

| | Model H: US liquid model |
|---|---|
| | detergent |
| Ingredient (Supplier) | (wt % of active ingredient) |
| LAS (Unger or Sasol/Danlind) | 4.70% |
| AEOS (Stepan/Stepan) | 5.90% |
| Coco fatty acid (Oleon/Danlind) | 2.00% |
| AEO (Stepan/Stepan) | 5.90% |
| MEA (Brenntag/Brenntag) | 1.50% |
| MPG (Brenntag/Danlind) | 2.00% |
| Ethanol (Brenntag/Danlind) | 1.00% |
| DTPA (Sigma-Aldrich) | 0.40% |
| Sodium citrate (Danlind) | 3.90% |
| Sodium formate (Sigma-Aldrich) | 0.20% |
| Sodium hydroxide (Solvay/Danlind) | 0.50% |
| Hydrochloric acid (Fluka) | 0.41% |
| Water, ion exchanged (Fluka) | 66.90% |

TABLE E
Model Detergent N composition

| | Model N: Asia pacific |
|---|---|
| | model detergent |
| Ingredient (Supplier) | (wt % of active ingredient) |
| LAS (Tianzhi Chemical) | 5.00% |
| AEOS (Tianzhi Chemical) | 10.70% |
| Soap | 1.00% |
| AEO (BASF) | 5.30% |
| Deionized water | 74.75% |
| TEA | 0.40% |
| Sodium hydroxide | 0.73% |
| Sodium citrate | 2.00% |
| CaCl₂•2H₂O | 0.02% |
| 5-chloro-2-methyl-4-isothiazolin- | 0.10% |
| 3-one and 2-methyl-4-isothiazolin- | |
| 3-one (Sunpu Biochemical) | |

TABLE F
Comercial Detergent Bluemoon composition

| | Bluemoon: Commercial detergent |
|---|---|
| Ingredient | (wt % of active ingredient) |
| LAS (Anion) | 3.60% |
| AEOS (Anion) | 12.95% |
| Soap | 1.50% |
| AEO (Nonion) | 3.30% |
| Deionized water | 76.96% |
| Glycerol | 0.10% |
| TEA | 0.00% |
| Na₂SO₄ | 0.17% |
| NaCl | 0.21% |
| Sodium citrate | 0.08% |
| FWA | 0.27% |

Persil Small & Mighty non-bio, Unilever, detergent registred February 2014, lot/batch 3316 1 P1 17:10, bought on www.britstore.co.uk
Persil Sensitive-Gel (1), Henkel, detergent registred September 2014, lot/batch 1743889-12-17254, bought on www.amazon.de
Persil Sensitive-Gel (2), Henkel, detergent registred January 2014, lot/batch 1743906-12-17254.

Wash Assay—

Terg-O-tometer (TOM) assay—The Tergotometer consists of sixteen 2 L metal beakers, each fitted with an agitator, which rotate in a back-and-forth manner at a controlled speed (120 rpm), to simulate the agitation mode occurring in commercial top-loader washing machines. The beakers are partly submerged in thermostatic water baths where the temperature can be controlled. Each beaker was filled with 1 L detergent solution and eight test swatches with ballast added up to 30 g (including test swatches). After a wash period of 20 minutes, the swatches were promptly removed from the beakers and rinsed thoroughly with running tap water for 6-7 minutes.

Drying of swatches—After washing and rinsing the swatches were placed in a rack with perforated metal plates covered with filtration paper and dried overnight at room temperature. For studying of the effect of drying, a drying step at 70° C. for 30 minutes was conducted prior to drying at room temperature.

Evaluation—Remission of the swatches was measured at 460 nm with Macbeth Color-Eye 7000 remission spectrophotometer 21 hours after wash. Remission values were calculated as the difference between reference vessel and sample (enzyme) vessel at the chosen wavelength: de_Rem=Remₛₐₘₚₗₑ−Rem_{ref}

The swatches were vacuum packed after evaluation if stored for further evaluation.

### Example 1

### Swatches with Colored Lipid Stains Comprising Different Lipase Substrates

TABLE 1A
Test conditions

| | |
|---|---|
| Detergent base | Model O |
| Detergent dosage | 2.0 g/L |
| Test solution volume | 1.0 L, TOM assay |
| pH of the detergent solution | 7.3 |
| Water hardness | 14°dH by addition of |
| | CaCl₂*2H₂O, MgCl₂*6H₂O (Ca²⁺:Mg²⁺ |
| | 3:2) |
| Wash time | 20 minutes |
| Temperature | 30° C. |
| Lipase provided by Novozymes | Lipex ® 100L |
| A/S, Bagsvaerd, Denmark | Lipolase ® 100L |
| Lipase concentration | 0.00; 0.05; 0.10; 0.20; 0.50 ppm |
| (mg protein pr L test solution) | |
| Swatches of the invention | Lard + beta-carotene + dl-alpha- |
| | tocopherol |
| | Lard/soybean oil + beta-carotene + |
| | dl-alpha-tocopherol |

TABLE 1B
Results

| | Lipase | Lard | | | Lard/Soybean oil | | |
|---|---|---|---|---|---|---|---|
| | dosage | Avg. | StDev | Delta | Avg. | StDev | Delta |
| Lipase | (ppm) | R460 | R460 | R460 | R460 | R460 | R460 |
| Lipex ® | 0.00 | 38.35 | 1.34 | 0.00 | 51.98 | 1.29 | 0.00 |
| 100L | 0.05 | 44.23 | 0.77 | 5.88 | 59.89 | 2.93 | 7.91 |
| | 0.10 | 47.60 | 1.05 | 9.25 | 61.07 | 2.38 | 9.09 |
| | 0.20 | 49.75 | 2.10 | 11.41 | 64.68 | 1.47 | 12.70 |
| | 0.50 | 58.25 | 1.52 | 19.90 | 65.93 | 2.52 | 13.95 |
| Lipolase ® | 0.00 | 37.70 | 0.71 | 0.00 | 51.56 | 1.93 | 0.00 |
| 100L | 0.05 | 38.31 | 1.17 | 0.61 | 51.71 | 1.15 | 0.16 |
| | 0.10 | 38.08 | 1.26 | 0.38 | 55.61 | 2.37 | 4.06 |
| | 0.20 | 39.18 | 3.10 | 1.48 | 56.37 | 2.17 | 4.81 |
| | 0.50 | 41.38 | 1.08 | 3.68 | 57.06 | 2.37 | 5.50 |
| | 0.75 | 43.68 | 1.55 | 5.98 | 56.56 | 1.57 | 5.00 |

### Example 2

### Swatch with Colored Lipid Stain Comprising Beef Fat/Peanut Oil as Lipase Substrate

TABLE 2A
Test conditions

| | |
|---|---|
| Detergent base | Model H |
| Detergent dosage | 1.5 g/L |
| Test solution volume | 1.0 L, TOM assay |
| pH of the detergent solution | 8.2 |
| Wash time | 20 minutes |
| Temperature | 20° C. |
| Water hardness | 6°dH |
| Lipase provided by Novozymes | Lipex ® 100L |
| A/S, Bagsvaerd, Denmark | |
| Lipase concentration | 0.00; 0.03; 0.05; 0.10; 0.25; 0.50; 0.75; |
| (mg protein pr L test solution) | 1.0 ppm |
| Swatches of the invention | Beef fat/peanut oil + beta-carotene + |
| | dl-alpha-tocopherol |

TABLE 2B
Results

| | Lipase | Beef fat/Peanut oil | | |
|---|---|---|---|---|
| | dosage | Avg. | StDev | Delta |
| Lipase | (ppm) | R460 | R460 | R460 |
| Lipex ® | 0.00 | 46.69 | 1.96 | 0.00 |
| 100L | 0.03 | 49.45 | 3.30 | 2.76 |
| | 0.05 | 50.71 | 2.47 | 4.02 |
| | 0.10 | 52.51 | 2.01 | 5.82 |
| | 0.25 | 59.27 | 1.61 | 12.58 |
| | 0.50 | 69.55 | 2.20 | 22.86 |
| | 0.75 | 69.78 | 2.54 | 23.09 |
| | 1.00 | 70.63 | 4.22 | 23.93 |

### Example 3

### Swatches with Colored Lipid Stains Comprising Different Lipase Substrates

TABLE 3A
Test conditions

| | |
|---|---|
| Detergent base | Model O |
| | Model N |
| Detergent dosage | 2 g/L |
| Test solution volume | Domestic washing machines (Panasonic, Japan) in Asian |
| | pacific region were used. They are top-loading washing |
| | machines with a water consumption of 33 L in the main |
| | washing step. |
| Washing time (main wash) | 15 minutes |
| Temperature | 25° C. |
| Water hardness | 14°dH |
| pH of the detergent solution | 7.8 in Model O |
| | 7.9 in Model N |
| Lipase provided by Novozymes | Experimental lipase |
| A/S, Bagsvaerd, Denmark | |
| Lipase concentration | 0.00; 0.05; 0.10; 0.20 ppm |
| (mg protein pr L test solution) | |
| Swatches of the invention | Lard + beta-carotene + dl-alpha-tocopherol |
| | Lard/Soybean oil + beta-carotene + dl-alpha-tocopherol |
| | Beef fat/Soybean oil + beta-carotene + dl-alpha-tocopherol |

TABLE 3B
Results with Model Detergent N

| | Lipase | Lard | | | Lard/Soybean oil | | | Beef fat/Soybean oil | | |
|---|---|---|---|---|---|---|---|---|---|---|
| | dosage | Avg. | StDev | Delta | Avg. | StDev | Delta | Avg. | StDev | Delta |
| Lipase | (ppm) | R460 | R460 | R460 | R460 | R460 | R460 | R460 | R460 | R460 |
| Exp. | 0.00 | 32.56 | 0.99 | 0.00 | 47.44 | 3.18 | 0.00 | 47.25 | 3.01 | 0.00 |
| lipase | 0.05 | 33.63 | 1.66 | 1.06 | 51.16 | 5.92 | 3.73 | 49.04 | 1.40 | 1.78 |
| | 0.10 | 35.12 | 1.93 | 2.55 | 51.03 | 4.18 | 3.59 | 52.28 | 3.56 | 5.03 |
| | 0.20 | 38.15 | 2.07 | 5.58 | 56.76 | 4.78 | 9.33 | 56.14 | 2.23 | 8.89 |

TABLE 3C
Results with Model Detergent O

| | Lipase | Lard | | | Lard/Soybean oil | | | Beef fat/Soybean oil | | |
|---|---|---|---|---|---|---|---|---|---|---|
| | dosage | Avg. | StDev | Delta | Avg. | StDev | Delta | Avg. | StDev | Delta |
| Lipase | (ppm) | R460 | R460 | R460 | R460 | R460 | R460 | R460 | R460 | R460 |
| Exp. | 0.00 | 32.09 | 2.51 | 0.00 | 44.41 | 3.47 | 0.00 | 43.33 | 3.07 | 0.00 |
| lipase | 0.10 | 35.67 | 1.89 | 3.58 | 50.41 | 4.58 | 6.00 | 49.14 | 2.31 | 5.80 |
| | 0.20 | 38.54 | 2.68 | 6.45 | 54.50 | 4.63 | 10.09 | 53.63 | 2.30 | 10.29 |

### Example 4

### Swatches with Colored Lipid Stains Comprising Tocopherol

TABLE 4A
Test conditions

| | |
|---|---|
| Detergent base | Bluemoon |
| Detergent dosage | 1.5 g/L |
| Test solution volume | 1.0 L, TOM assay |
| pH of the detergent solution | 7.0 |
| Water hardness | 14°dH by addition of CaCl₂*2H₂O, MgCl₂*6H₂O (Ca²⁺:Mg²⁺ |
| | 3:2) |
| Wash time | 20 minutes |
| Temperature | 30° C. |
| Lipase provided by Novozymes | Lipex ® 100L |
| A/S, Bagsvaerd, Denmark | |
| Lipase concentration | 0.00; 0.05; 0.10; 0.30 ppm |
| (mg protein pr L test solution) | |
| Swatches of the invention | Lard + beta-carotene |
| | Lard + beta-carotene + dl-alpha-tocopherol |
| | Lard/Soybean oil + beta-carotene |
| | Lard/Soybean oil + beta-carotene + dl-alpha-tocopherol |

TABLE 4B
Results

| | Lipase | Lard | | | Lard + tocopherol | | |
|---|---|---|---|---|---|---|---|
| | dosage | Avg. | StDev | Delta | Avg. | StDev | Delta |
| Lipase | (ppm) | R460 | R460 | R460 | R460 | R460 | R460 |
| Lipex ® | 0.00 | 41.46 | 2.06 | 0.00 | 36.69 | 1.22 | 0.00 |
| 100L | 0.05 | 48.75 | 3.59 | 7.29 | 40.47 | 1.11 | 3.78 |
| | 0.10 | 56.45 | 2.23 | 14.99 | 42.62 | 1.64 | 5.93 |
| | 0.30 | 73.05 | 2.55 | 31.59 | 48.33 | 0.94 | 11.64 |
| | | | | | Lard/soybean oil + | | |
| | Lipase | Lard/soybean oil | | | tocopherol | | |
| | dosage | Avg. | StDev | Delta | Avg. | StDev | Delta |
| Lipase | (ppm) | R460 | R460 | R460 | R460 | R460 | R460 |
| Lipex ® | 0.00 | 49.09 | 2.24 | 0.00 | 44.21 | 1.31 | 0.00 |
| 100L | 0.05 | 58.02 | 0.82 | 8.93 | 51.06 | 0.82 | 6.85 |
| | 0.10 | 60.56 | 0.65 | 11.47 | 55.22 | 1.38 | 11.01 |
| | 0.30 | 64.39 | 1.52 | 15.30 | 58.68 | 1.65 | 14.47 |

### Example 5

### Swatches with Colored Lipid Stains Comprising Various Amounts of Tocopherol

TABLE 5A
Test conditions

| | |
|---|---|
| Detergent base | Model H |
| Detergent dosage | 1.5 g/L |
| Test solution volume | 1.0 L, TOM assay |
| pH of the detergent solution | 8.2 |
| Water hardness | 6°dH |
| Wash time | 20 minutes |
| Temperature | 20° C. |
| Lipase provided by Novozymes | Lipex ® 100L |
| A/S, Bagsvaerd, Denmark | |
| Lipase concentration | 0.00; 0.05; 0.10; 0.20; 0.30 ppm |
| (mg protein pr L test solution) | |
| Swatches of the invention | Lard + beta-carotene |
| | Lard + beta-carotene + 0.02% |
| | dl-alpha-tocopherol |
| | Lard + beta-carotene + 0.04% |
| | dl-alpha-tocopherol |

TABLE 5B
Results

| | | | | | Lard + beta-carotene + | | | Lard + beta-carotene + | | |
|---|---|---|---|---|---|---|---|---|---|---|
| | Lipase | Lard + beta-carotene | | | 0.02% tocopherol | | | 0.04% tocopherol | | |
| | dosage | Avg. | StDev | Delta | Avg. | StDev | Delta | Avg. | StDev | Delta |
| Lipase | (ppm) | R460 | R460 | R460 | R460 | R460 | R460 | R460 | R460 | R460 |
| Lipex ® | 0.00 | 46.20 | 2.27 | 0.00 | 35.36 | 0.77 | 0.00 | 33.94 | 0.47 | 0.00 |
| 100L | 0.05 | 55.19 | 4.35 | 8.99 | 39.65 | 0.75 | 4.29 | 39.41 | 1.34 | 5.47 |
| | 0.10 | 59.08 | 2.47 | 12.87 | 44.32 | 1.18 | 8.97 | 41.28 | 0.87 | 7.34 |
| | 0.20 | 64.27 | 3.70 | 18.07 | 45.17 | 0.42 | 9.81 | 44.62 | 0.88 | 10.68 |
| | 0.30 | 72.53 | 5.49 | 26.33 | 47.32 | 0.94 | 11.96 | — | — | — |

### Example 6

### Swatches with Colored Lipid Stains Comprising Various Amounts of Substrate

TABLE 6A
Test conditions

| | |
|---|---|
| Detergent base | Model O |
| Detergent dosage | 2 g/L |
| Test solution volume | 1.0 L, TOM assay |
| pH of the detergent solution | 7.4 |
| Water hardness | 14°dH by addition of CaCl₂*2H₂O, |
| | MgCl₂*6H₂O (Ca²⁺:Mg²⁺ 3:2) |
| Wash time | 20 minutes |
| Temperature | 30° C. |
| Lipase provided | Lipex ® 100L |
| by Novozymes | |
| A/S, Bagsvaerd, Denmark | |
| Lipase concentration | 0.00; 0.05; 0.10; 0.20 ppm |
| (mg protein pr L test solution) | |
| Swatches of the invention | 0.1 mL Lard + beta-carotene + tocopherol |
| | 0.2 mL Lard + beta-carotene + tocopherol |
| | 0.3 mL Lard + beta-carotene + tocopherol |
| | 0.4 mL Lard + beta-carotene + tocopherol |

TABLE 6B
Results

| | Lipase | 0.1 mL Lard | | | 0.2 mL Lard | | |
|---|---|---|---|---|---|---|---|
| | dosage | Avg. | StDev | Delta | Avg. | StDev | Delta |
| Lipase | (ppm) | R460 | R460 | R460 | R460 | R460 | R460 |
| exp. | 0.00 | 45.54 | 1.51 | 0.00 | 44.15 | 1.39 | 0.00 |
| Lipase | 0.05 | 49.83 | 2.11 | 4.30 | 50.03 | 1.25 | 5.87 |
| | 0.10 | 54.74 | 1.79 | 9.20 | 54.40 | 1.27 | 10.25 |
| | 0.20 | 61.20 | 1.11 | 15.66 | 59.34 | 0.77 | 15.19 |
| | Lipase | 0.3 mL Lard | | | 0.4 mL Lard | | |
| | dosage | Avg. | StDev | Delta | Avg. | StDev | Delta |
| Lipase | (ppm) | R460 | R460 | R460 | R460 | R460 | R460 |
| exp. | 0.00 | 40.72 | 0.48 | 0.00 | 38.27 | 0.75 | 0.00 |
| Lipase | 0.05 | 45.79 | 0.14 | 5.08 | 42.35 | 0.60 | 4.08 |
| | 0.10 | 49.79 | 0.40 | 9.07 | 46.66 | 0.67 | 8.39 |
| | 0.20 | 53.89 | 0.95 | 13.17 | 51.09 | 1.06 | 12.83 |

### Example 7

### Swatches of the Invention Compared with Commercial Swatches

TABLE 7A
Test conditions

| | |
|---|---|
| Detergent base | Model O |
| Detergent dosage | 2 g/L |
| Test solution volume | 1.0 L, TOM assay |
| pH of the detergent solution | 7.4 |
| Water hardness | 14°dH by addition of CaCl₂*2H₂O, |
| | MgCl₂*6H₂O (Ca²⁺:Mg²⁺ 3:2) |
| Wash time | 20 minutes |
| Temperature | 30° C. |
| Lipase provided by Novozymes | Lipex ® 100L |
| A/S, Bagsvaerd, Denmark | |
| Lipase concentration | 0.00; 0.05; 0.10; 0.20 ppm |
| (mg protein pr L test solution) | |
| Swatches of the invention | Lard + beta-carotene + |
| | dl-alpha-tocopherol |
| Commercial swatches | C-S-61, Beef fat, colored |
| | C-S-64, Beef fat, soot |
| | C-S-62, Lard, colored |
| | C-04, Chlorophyl, Vegetable Oil |
| | W-10PF, Pigment/Vegetable Fat |
| | W-20PF, Pigment/Vegetable Fat |

TABLE 7
Results

| | Lipase | Lard | | |
|---|---|---|---|---|
| | dosage | Avg. | StDev | Delta |
| Lipase | (ppm) | 460 | 460 | R460 |
| Lipex ® | 0.00 | 44.15 | 1.39 | 0.00 |
| 100L | 0.05 | 50.03 | 1.25 | 5.87 |
| | 0.10 | 54.40 | 1.27 | 10.25 |
| | 0.20 | 59.34 | 0.77 | 15.19 |

| | Lipase | C-S-61 Beef fat/colored | | | C-S-64 Beef fat/soot | | | C-S-62 Lard/colored | | |
|---|---|---|---|---|---|---|---|---|---|---|
| | dosage | Avg. | StDev | Delta | Avg. | StDev | Delta | Avg. | StDev | Delta |
| Lipase | (ppm) | 460 | 460 | R460 | 460 | 460 | R460 | 460 | 460 | R460 |
| Lipex ® | 0.00 | 40.58 | 2.94 | 0.00 | 25.84 | 1.54 | 0.00 | 38.14 | 0.97 | 0.00 |
| 100L | 0.05 | 47.63 | 3.83 | 7.05 | 26.09 | 1.44 | 0.26 | 37.78 | 0.97 | −0.37 |
| | 0.10 | 47.06 | 3.09 | 6.48 | 26.16 | 0.99 | 0.33 | 37.07 | 1.44 | −1.07 |
| | 0.20 | 48.61 | 2.18 | 8.03 | 25.74 | 1.04 | −0.10 | 39.14 | 2.50 | 1.00 |
| | | C-04 | | | W-10PF | | | W-20PF | | |
| | Lipase | Chlorophyl/Vegetable oil | | | Pigment/Vegetable Fat | | | Pigment/Vegetable Fat | | |
| | dosage | Avg. | StDev | Delta | Avg. | StDev | Delta | Avg. | StDev | Delta |
| Lipase | (ppm) | 460 | 460 | R460 | 460 | 460 | R460 | 460 | 460 | R460 |
| Lipex ® | 0.00 | 45.98 | 0.44 | 0.00 | 53.13 | 0.92 | 0.00 | 64.69 | 0.61 | 0.00 |
| 100L | 0.05 | 47.01 | 0.38 | 1.03 | 56.81 | 1.72 | 3.68 | 67.64 | 0.24 | 2.95 |
| | 0.10 | 46.90 | 0.65 | 0.92 | 57.59 | 1.10 | 4.46 | 69.00 | 0.53 | 4.31 |
| | 0.20 | 46.41 | 0.33 | 0.43 | 58.23 | 1.58 | 5.10 | 68.68 | 0.50 | 3.99 |

### Example 8

### Swatches of the Invention Compared with Commercial Swatches in Full Scale Wash with Different Model Detergents

TABLE 8A
Test conditions

| | |
|---|---|
| Detergent base | Model N |
| | Model O |
| Detergent dosage | 2 g/L |
| Test solution volume | Domestic washing machines (Panasonic, Japan) in Asian |
| | pacific region were used. They are top-loading washing |
| | machines with a water consumption of 33 L in the main |
| | washing step |
| pH of the detergent solution | 7.8 in Model O |
| | 7.9 in Model N |
| Water hardness | 14°dH |
| Wash time | 15 minutes |
| Temperature | 25° C. |
| Lipase provided by Novozymes | Experimental lipase |
| A/S, Bagsvaerd, Denmark | |
| Lipase concentration | 0.00; 0.05; 0.10; 0.20 ppm |
| (mg protein pr L test solution) | |
| Swatches of the invention | Lard + beta-carotene + dl-alpha-tocopherol |
| (4 pieces pr each beaker) | Lard/Soybean oil + beta-carotene + dl-alpha-tocopherol |
| | Beef fat + beta-carotene + dl-alpha-tocopherol |
| | Beef fat/Soybean oil + beta-carotene + dl-alpha-tocopherol |
| Commercial swatches | KC-S-62 Lard |
| | KC-S-78 Soybean oil |
| | KC-S-61 Beef fat |

TABLE 8B
Results with Model Detergent N

| | Lipase | Lard | | | Lard/Soybean oil | | |
|---|---|---|---|---|---|---|---|
| | dosage | Avg. | StDev | Delta | Avg. | StDev | Delta |
| Lipase | (ppm) | 460 | 460 | R460 | 460 | 460 | R460 |
| exp. | 0.00 | 32.56 | 0.99 | 0.00 | 47.44 | 3.18 | 0.00 |
| Lipase | 0.05 | 33.63 | 1.66 | 1.06 | 51.16 | 5.92 | 3.73 |
| | 0.10 | 35.12 | 1.93 | 2.55 | 51.03 | 4.18 | 3.59 |
| | 0.20 | 38.15 | 2.07 | 5.58 | 56.76 | 4.78 | 9.33 |
| | Lipase | Beef fat | | | Beef fat/Soybean oil | | |
| | dosage | Avg. | StDev | Delta | Avg. | StDev | Delta |
| Lipase | (ppm) | 460 | 460 | R460 | 460 | 460 | R460 |
| exp. | 0.00 | 40.92 | 3.97 | 0.00 | 47.25 | 3.01 | 0.00 |
| Lipase | 0.05 | 45.50 | 7.34 | 4.58 | 49.04 | 1.40 | 1.78 |
| | 0.10 | 43.75 | 5.13 | 2.83 | 52.28 | 3.56 | 5.03 |
| | 0.20 | 49.46 | 6.42 | 8.53 | 56.14 | 2.23 | 8.89 |

| | Lipase | KC-S-62 lard | | | KC-S-78 soybean oil | | | KC-S-61 beef fat | | |
|---|---|---|---|---|---|---|---|---|---|---|
| | dosage | Avg. | StDev | Delta | Avg. | StDev | Delta | Avg. | StDev | Delta |
| Lipase | (ppm) | 460 | 460 | R460 | 460 | 460 | R460 | 460 | 460 | R460 |
| exp. | 0.00 | 25.34 | 2.19 | 0.00 | 66.83 | 0.59 | 0.00 | 34.81 | 2.48 | 0.00 |
| Lipase | 0.05 | 25.58 | 1.98 | 0.23 | 68.44 | 0.86 | 1.61 | 33.44 | 1.80 | −1.37 |
| | 0.10 | 27.03 | 1.51 | 1.68 | 68.40 | 1.55 | 1.57 | 36.37 | 1.48 | 1.55 |
| | 0.20 | 30.14 | 0.87 | 4.80 | 68.97 | 0.44 | 2.14 | 39.66 | 2.36 | 4.85 |

TABLE 8C
Results with Model Detergent O

| | Lipase | Lard | | | Lard/Soybean oil | | |
|---|---|---|---|---|---|---|---|
| | dosage | Avg. | StDev | Delta | Avg. | StDev | Delta |
| Lipase | (ppm) | 460 | 460 | R460 | 460 | 460 | R460 |
| exp. | 0.00 | 32.09 | 2.51 | 0.00 | 44.41 | 3.47 | 0.00 |
| Lipase | 0.10 | 35.67 | 1.89 | 3.58 | 50.41 | 4.58 | 6.00 |
| | 0.20 | 38.54 | 2.68 | 6.45 | 54.50 | 4.63 | 10.09 |
| | Lipase | Beef fat | | | Beef fat/Soybean oil | | |
| | dosage | Avg. | StDev | Delta | Avg. | StDev | Delta |
| Lipase | (ppm) | 460 | 460 | R460 | 460 | 460 | R460 |
| exp. | 0.00 | 36.04 | 2.48 | 0.00 | 43.33 | 3.07 | 0.00 |
| Lipase | 0.10 | 41.28 | 3.16 | 5.25 | 49.14 | 2.31 | 5.80 |
| | 0.20 | 49.94 | 8.47 | 13.91 | 53.63 | 2.30 | 10.29 |

| | Lipase | KC-S-62 lard | | | KC-S-78 soybean oil | | | KC-S-61 beef fat | | |
|---|---|---|---|---|---|---|---|---|---|---|
| | dosage | Avg. | StDev | Delta | Avg. | StDev | Delta | Avg. | StDev | Delta |
| Lipase | (ppm) | 460 | 460 | R460 | 460 | 460 | R460 | 460 | 460 | R460 |
| exp. | 0.00 | 22.61 | 1.62 | 0.00 | 67.58 | 0.87 | 0.00 | 30.46 | 0.76 | 0.00 |
| Lipase | 0.10 | 26.05 | 0.70 | 3.44 | 68.99 | 0.58 | 1.41 | 34.13 | 0.81 | 3.68 |
| | 0.20 | 28.00 | 0.54 | 5.40 | 68.81 | 1.19 | 1.23 | 36.08 | 1.21 | 5.62 |

### Example 9

### Effect of Different Drying Conditions after Washing of Swatches of the Invention

TABLE 9A
Test conditions

| | |
|---|---|
| Detergent base | Bluemoon |
| Detergent dosage | 1.5 g/L |
| Test solution volume | 1.0 L, TOM assay |
| pH of the detergent solution | 7.2 |
| Water hardness | 14°dH by addition of CaCl₂*2H₂O, |
| | MgCl₂*6H₂O (Ca²⁺:Mg²⁺ 3:2) |
| Wash time | 20 minutes |
| Temperature | 30° C. |
| Lipase provided by Novozymes | Lipex ® 100L |
| A/S, Bagsvaerd, Denmark | |
| Lipase concentration | 0.00; 0.05; 0.10; 0.30 ppm |
| (mg protein pr L test solution) | |
| Swatches of the invention | Lard + beta-carotene |
| | Lard + beta-carotene + |
| | dl-alpha-tocopherol |

TABLE 9B
Results without tocopherol

| | | | | | Drying at 70° C. for 30 | | |
|---|---|---|---|---|---|---|---|
| | | Drying at 70° C. for 30 | | | minutes + 20.5 hours at | | |
| | Lipase | minutes | | | room temperature | | |
| | dosage | Avg. | StDev | Delta | Avg. | StDev | Delta |
| Lipase | (ppm) | 460 | 460 | R460 | 460 | 460 | R460 |
| Lipex ® | 0.00 | 35.99 | 0.30 | 0.00 | 44.65 | 1.86 | 0.00 |
| 100L | 0.05 | 40.04 | 0.52 | 4.05 | 56.28 | 2.81 | 11.63 |
| | 0.10 | 42.28 | 0.29 | 6.29 | 67.35 | 2.74 | 22.70 |
| | 0.30 | 44.98 | 0.77 | 8.98 | 80.10 | 1.15 | 35.44 |

| | | Drying at room | | | Drying at room | | |
|---|---|---|---|---|---|---|---|
| | | temperature for 21 | | | temperature for 24 | | |
| | Lipase | hours | | | hours | | |
| | dosage | Avg. | StDev | Delta | Avg. | StDev | Delta |
| Lipase | (ppm) | 460 | 460 | R460 | 460 | 460 | R460 |
| Lipex ® | 0.00 | 45.46 | 1.12 | 0.00 | 46.88 | 0.77 | 0.00 |
| 100L | 0.05 | 53.69 | 1.27 | 8.22 | 58.16 | 1.79 | 11.28 |
| | 0.10 | 56.97 | 2.70 | 11.50 | 63.26 | 2.80 | 16.38 |
| | 0.30 | 69.77 | 2.86 | 24.31 | 75.66 | 0.41 | 28.78 |

TABLE 9C
Results with tocopherol

| | | | | | Drying at 70° C. for 30 | | |
|---|---|---|---|---|---|---|---|
| | | Drying at 70° C. for 30 | | | minutes + 20.5 hours at | | |
| | Lipase | minutes | | | room temperature | | |
| | dosage | Avg. | StDev | Delta | Avg. | StDev | Delta |
| Lipase | (ppm) | 460 | 460 | R460 | 460 | 460 | R460 |
| Lipex ® | 0.00 | 36.81 | 0.57 | 0.00 | 40.93 | 0.92 | 0.00 |
| 100L | 0.05 | 40.87 | 0.88 | 4.06 | 45.06 | 0.71 | 4.14 |
| | 0.10 | 42.41 | 0.55 | 5.59 | 48.08 | 0.96 | 7.15 |
| | 0.30 | 46.33 | 0.92 | 9.52 | 54.21 | 0.48 | 13.28 |

| | | Drying at room | | | Drying at room | | |
|---|---|---|---|---|---|---|---|
| | | temperature for 21 | | | temperature for 24 | | |
| | Lipase | hours | | | hours | | |
| | dosage | Avg. | StDev | Delta | Avg. | StDev | Delta |
| Lipase | (ppm) | 460 | 460 | R460 | 460 | 460 | R460 |
| Lipex ® | 0.00 | 40.41 | 0.42 | 0.00 | 41.06 | 0.60 | 0.00 |
| 100L | 0.05 | 45.18 | 1.07 | 4.77 | 46.42 | 1.09 | 5.36 |
| | 0.10 | 47.15 | 1.38 | 6.74 | 48.28 | 0.80 | 7.22 |
| | 0.30 | 51.78 | 0.57 | 11.37 | 55.13 | 1.42 | 14.07 |

### Example 10

### Swatches with Lycopene Colored Lipid Stains Comprising Various Amounts of Tocopherol

TABLE 10A
Test conditions

| | |
|---|---|
| Detergent base | Model O |
| Detergent dosage | 2.0 g/L |
| Test solution volume | 1.0 L, TOM assay |
| pH of the detergent solution | 7.8 |
| Water hardness | 14°dH by addition of CaCl₂*2H₂O, |
| | MgCl₂*6H₂O (Ca²⁺:Mg²⁺ 3:2) |
| Wash time | 20 minutes |
| Temperature | 30° C. |
| Lipase provided by Novozymes | Lipex ® 100L |
| A/S, Bagsvaerd, Denmark | |
| Lipase concentration | 0.00; 0.05; 0.10; 0.30 ppm |
| (mg protein pr L test solution) | |
| Swatches of the invention | Lard + lycopene |
| | Lard + lycopene + 0.01% |
| | dl-alpha-tocopherol |
| | Lard + lycopene + 0.02% |
| | dl-alpha-tocopherol |
| | Lard + lycopene + 0.04% |
| | dl-alpha-tocopherol |

TABLE 10B
Results

| | | | | | Lard + lycopene + | | |
|---|---|---|---|---|---|---|---|
| | | | | | 0.01% dl-alpha- | | |
| | Lipase | Lard + lycopene | | | tocopherol | | |
| | dosage | Avg. | StDev | Delta | Avg. | StDev | Delta |
| Lipase | (ppm) | R460 | R460 | R460 | R460 | R460 | R460 |
| Lipex ® | 0.00 | 62.59 | 0.67 | 0.00 | 62.82 | 2.21 | 0.00 |
| 100L | 0.05 | 68.18 | 0.21 | 5.59 | 64.80 | 0.65 | 1.98 |
| | 0.10 | 72.86 | 1.92 | 10.27 | 67.20 | 0.95 | 4.38 |
| | 0.30 | 76.68 | 0.18 | 14.09 | 71.65 | 0.79 | 8.83 |
| | | Lard + lycopene + | | | Lard + lycopene + | | |
| | | 0.02% dl-alpha- | | | 0.04% dl-alpha- | | |
| | Lipase | tocopherol | | | tocopherol | | |
| | dosage | Avg. | StDev | Delta | Avg. | StDev | Delta |
| Lipase | (ppm) | R460 | R460 | R460 | R460 | R460 | R460 |
| Lipex ® | 0.00 | 59.78 | 0.59 | 0.00 | 58.74 | 1.69 | 0.00 |
| 100L | 0.05 | 62.20 | 0.48 | 2.42 | 61.24 | 0.97 | 2.49 |
| | 0.10 | 66.54 | 0.64 | 6.76 | 67.11 | 1.01 | 8.37 |
| | 0.30 | 69.47 | 0.97 | 9.70 | 71.31 | 1.54 | 12.57 |

### Example 11

### Swatches with Colored Lipid Stains Comprising Lycopene as Color

TABLE 11A
Test conditions

| | |
|---|---|
| Detergent base | Persil Small & Mighty non-bio |
| Detergent dosage | 2.3 g/L |
| Test solution | 15 L FSW |
| volume | |
| Wash program | Cotton/Koge-/kulørt vask, Miele Softtronic W2445, |
| | wash time: 1 h 23 min (mainwash 43-45 min) |
| Temperature | 30° C. |
| Water hardness | 15°dH |
| Lipase | Lipex ® Evity ® 100L |
| Lipase concentration | 0 wt %, 0.12 wt %, 0.3 wt % |
| Swatches of the | Lard + Lycopene + dl-alpha-tocopherol |
| invention | |

TABLE 11B
Results

| | Lipase | Lard + Lycopene + dl-alpha-tocopherol | | |
|---|---|---|---|---|
| Lipase | dosage | Avg R460 | StDev R460 | Delta R460 |
| Lipex ® Evity ® | 0.00 wt % | 62.37 | 2.6 | 0 |
| 100L | 0.12 wt % | 69.46 | 1.9 | 7.09 |
| | 0.3 wt % | 73.57 | 2.5 | 11.20 |

### Example 12

### Swatches with Colored Lipid Stains Comprising Tocopherol and Chili as Color

TABLE 12A
Test conditions

| | |
|---|---|
| Detergent base | Persil Sensitive-Gel (1) |
| Detergent dosage | 4.9 g/L |
| Test solution volume | 1.0 L TOM assay (120 rpm) |
| pH of the detergent solution | 8.89 |
| Wash time | 20 minutes |
| Temperature | 20° C. |
| Water hardness | 15°dH |
| Lipase | Lipex ® 100L |
| Lipase concentration | 0 ppm, 0.1 ppm, 0.3 ppm, 0.5 ppm |
| Swatches of the invention | Lard + Chili + dl-alpha-tocopherol |

TABLE 12B
Results

| | | Lard + Chili + dl-alpha-tocopherol | | |
|---|---|---|---|---|
| Lipase | Lipase dosage | Avg R460 | StDev R460 | Delta R460 |
| Lipex ® 100L | 0.0 ppm | 62.40 | 0.05 | 0 |
| Lipex ® 100L | 0.1 ppm | 66.23 | 1.87 | 3.83 |
| Lipex ® 100L | 0.2 ppm | 70.93 | 0.26 | 8.53 |
| Lipex ® 100L | 0.3 ppm | 71.27 | 2.72 | 8.87 |

### Example 13

### Swatches with Colored Lipid Stains Comprising Paprika as Color

TABLE 13A
Test conditions

| | |
|---|---|
| Detergent base | Persil Sensitive-Gel (2) |
| Detergent dosage | 4.9 g/L |
| Test solution volume | 1.0 L TOM assay (120 rpm) |
| Wash time | 30 minutes |
| Temperature | 30° C. |
| Water hardness | 15°dH |
| Lipase | Lipex ® Evity ® 100L (Non-commercial |
| | batch) |
| Lipase concentration | 0.0 wt %, 0.2 wt %, 0.6 wt % |
| Swatches of the invention | Lard + Paprika |

TABLE 13B
Results

| | | Lard + paprika + | | |
|---|---|---|---|---|
| | | dl-alpha-tocopherol | | |
| | Lipase | | | Delta |
| Lipase | dosage | Avg R460 | StDev R460 | R460 |
| Lipex ® Evity ® 100L | 0.0 wt % | 43.59 | 0.25 | 0 |
| Lipex ® Evity ® 100L | 0.2 wt % | 52.95 | 0.20 | 9.4 |
| Lipex ® Evity ® 100L | 0.6 wt % | 59.3 | 0.21 | 15.7 |

1. A swatch comprising a fabric and a colored lipid stain, wherein said colored lipid stain comprises one or more lipase substrates, at least one carotenoid, and optionally a color preserving agent.

2. The swatch according to claim 1 wherein the fabric is selected from natural plant fibres, animal based fibres, synthetic fibres, or any combinations thereof.

3. The swatch according to claim 1 wherein the fabric is knitted or woven.

4. The swatch according to claim 1 wherein the one or more e.g. several lipase substrates are selected from oils, fats, or any combination thereof.

5. The swatch according to claim 4 wherein fat may be lard, tallow, beef fat, milk fat, butter, or free milk fat; oils may be coconut oil, palm oil, olive oil, sesame oil, cottonseed oil, sunflower oil, corn oil, safflower oil, canola oil, soybean oil, fish oil terpenoid and essential oils; or any combination thereof.

6. The swatch according to claim 1 wherein the at least one carotenoid is selected from lutein, zeaxanthin, alpha-carotene, beta-carotene, lycopene, astaxanthin, canthaxanthin, phytoene, apo carotenal, retinol, capsanthin, zeaxanthin, beta-cryptoxanthin, phytofluene or any mixtures thereof

7. The swatch according to claim 1 wherein the color preserving agent is an anti-oxidant.

8. The swatch according to claim 1 wherein the anti-oxidant is selected from tocopherols e.g. dl-alpha-tocopherol, Vitamin C, Ascorbyl palmitate, or any combination thereof.

9. Method for preparing the swatch according to claim 1 comprising: (a) applying a colored lipid stain to a fabric; (b) heating the swatch to a temperature above the melting temperature of the lipase substrate(s).

10. (canceled)

11. Method for testing wash performance of an enzyme with lipase activity comprising:
(a) contacting the swatch according to claim 1 with a detergent composition comprising said enzyme;
(b) rinsing the swatch with water;
(c) drying the swatch; and
(d) measuring the color intensity of the swatch at one or more wavelengths; and
comparing said color intensity with the color intensity of a control swatch.

12. A colored lipid stain comprising one or more lipase substrates, at least one coloring agent and optionally a color preserving agent, wherein the one or more lipase substrates constitute more than 20 wt % based on the weight of the colored lipid stain.

13. The colored lipid stain according to claim 12, wherein the at least one coloring agent and/or the optionally color preserving agent is individually present in concentrations from 0.001 wt % up to and including 0.4 wt % based on the weight of the colored lipid stain.

14. The colored lipid stain according to claim 1, wherein the at least one coloring agent is a carotenoid selected from lutein, zeaxanthin, alpha-carotene, beta-carotene, lycopene, astaxanthin, canthaxanthin, phytoene, apo carotenal, retinol, capsanthin, zeaxanthin, beta-cryptoxanthin, phytofluene or any mixtures thereof.

15. The colored lipid stain according to claim 1, wherein the color preserving agent is an anti-oxidant selected from tocopherols Vitamin C, Ascorbyl palmitate, or any combination thereof.


SEQUENCE LISTING (1 sequence)

SEQ ID NO: 1 - protein, 269 aa, Unknown [sequence omitted]